(19) United States
(12) Patent Application Publication
(10) Pub. No.: US 2008/0112982 A1
(43) Pub. Date: May 15, 2008

(54) METHODS AND COMPOSITIONS RELATED TO BACTERIAL FLAGELLUM AND NANOTUBE FORMATION

(76) Inventor: Kelly T. Hughes, Salt Lake City, UT (US)

Correspondence Address: NEEDLE & ROSENBERG, P.C., SUITE 1000, 999 PEACHTREE STREET, ATLANTA, GA, 30309-3915, US

(21) Appl. No.: 11/761,866

(22) Filed: Jun. 12, 2007

Related U.S. Application Data
(60) Provisional application No. 60/812,784, filed on Jun. 12, 2006.

(51) Int. Cl. A61K 39/02; C12N 1/21; C07K 14/195

(52) U.S. Cl. 424/242.1; 435/252.3; 530/350; 977/906

(57) ABSTRACT

Disclosed are compositions and methods relating to bacterial flagella and uses thereof.

### CROSS REFERENCE TO RELATED APPLICATIONS

This application claims the benefit of priority of U.S. Provisional Application No. 60/812,784, filed Jun. 12, 2006, which application is incorporated herein by this reference in its entirety.

### ACKNOWLEDGEMENTS

This invention was made with government support under NIH Grant GM56141. The government has certain rights in this invention.

### BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows flagellar assembly and regulatory pathways of Salmonella typhimurium. A. A schematic of the flagellar assembly pathway. B. The flagellar transcriptional hierarchy is coupled to flagellar morphogenesis by secretion of FlgM through the completed hook-basal body.

FIG. 2 shows the flgG regulatory mutants result in a polyrod phenotype. A. An electron micrograph of isolated HBB structures from (A.) wild-type cells (SJW1103) and (B.) from the ring-defective (ΔflgHI) flgG regulatory (flgG*) double mutants (TH5931). C. The rod-length measurements of isolated flagellar basal structures from a ring mutant strain with a FlgG*-rod (TH5931). D. Excess FlgG is present in the polyrod structures. The composition of basal structure rod subunits along with the MS-ring subunit, FliF, in wild-type (SJW1103), polyrod (TH5931, FlgG*-rod) and super-polyrod (TH9709, ΔfliK FlgG*-rod) producing strains by SDS-PAGE. Loading was controlled by determining concentration of FliF in each fraction and loading equal amounts of FliF.

FIG. 3 shows the removal of FliK in the polyrod strains results in the super-polyrod phenotype. A. Isolated polyrods from strain TH9709 (ΔflgHI flgG* ΔfliK) without associated polyhooks. B. A polyrod polyhook structure from strain TH9709. C. Extension of the super-polyrod through the outer membrane of the cell in osmotically shocked strain TH9709.

FIG. 4 shows the rod length distribution in strain TH9709 (ΔflgHI flgG* ΔfliK). A. The rod length distribution of super-polyrod structures that did not have associated polyhooks and representative polyrod structures. B. The rod length distribution of super-polyrods that had attached polyhooks and representative structures.

FIG. 5 shows the effect of FliK deletions in the proline-rich region on hook-length control. Twenty six deletions in the proline-rich region of FliK were constructed and their effect on hook length were determined. The majority resulted in the FliK null phenotype (polyhook). However, three regions including amino acids 121 through 133, 161 through 202, and 238 through 278 were identified that could be removed and resulted in shorter hook structures.

FIG. 6 shows modeling FlgG and polyrod mutants on the 3D structure of FlgE (hook). A. Alignment of FlgG-rod and FlgE-hook sequences. The FlgG-rod protein is 260 amino acids in length. Of these, 38% correspond to identical residues (in red) in the corresponding FlgE sequences throughout the FlgG protein, while the predicted structural conservation (blue for FlgE and light-blue for FlgG) is nearly identical. The exceptions are a large insertion in FlgE-hook relative to FlgG that defines a complete and separate domain from the core of the FlgE protein (Samatey et al., 2004). In addition FlgG contains an 18 amino acid insertion after the corresponding residue 43 of FlgE. This region includes the majority of the FlgG changes that are defective in the stop-polymerization mechanism (shaded in green). B. Mutational changes in FlgG that result in the polyrod phenotype. Single amino acid substitutions are shown with a single arrow pointing from the original base. For some codons multiple single amino acid substitutions were obtained. These are shown stacked on one another. Three deletions are depicted with multiple arrows that fuse to a point. They are: an in-frame deletion of codons A54-Q55-S56-S56, a deletion that changes codons Q59-T60-T61-L62-P63 to a single histidine codon, and deletion of codons G65-L66. The A symbol was used to denote a deletion. C. A FlgG structural model based on FlgE. A 3-dimensional structure of FlgG based on the crystal structure of FlgE. The FlgE crystal structure is missing the amino and carboxy-terminal regions corresponding to residues 1 through 91 from the N-terminus of FlgG and residues 223 through 260 from the C-terminus and thus these regions could not be included in the model. The region including the majority of FlgG* mutations (residues 52 through 66 drawn in a green oval) are located near the top of the FlgG subunit where it interacts with residues G183 and S197, located at the bottom of the structure during FlgG polymerization.

FIG. 7 shows flagellar structures from a flgG* mutant strains. A. Intact filament structures were isolated from a strain (TH9614) that carried a single flgG* allele (flgG*5664 (G53C)) and examined by electron microscopy. The two smaller structures visible in the left micrograph are virulence-associated type III needle structures that co-purify with flagellar filaments. B. Intact filaments were isolated from a strain (TH9616) that carried a single flgG* allele (flgG*5671 (P52L)) except that filaments were depolymerized by acid treatment and the final flagellar basal structures examined by electron microscope.

FIG. 8 shows growth of flagella in the periplasm in flgG* mutant strains. Strains that carry only a flgG* allele (TH9613 (flgG*5662 (G65E)) (top) and TH10080 (flgG*5670 (G183R)) (bottom) were subject to lysozyme treatment in order to visualize flagellar structures growing in the periplasmic space by electron microscopy. Occasionally, membrane-enveloped flagellar filaments structures were observed (bottom).

### DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

The term “set” refers to a collection of one or more elements. Thus, for example, a set of nanostructures may comprise a single nanostructure or multiple nanostructures. Elements of a set can also be referred to as members of the set. Elements of a set can be the same or different. In some instances, elements of a set can share one or more common characteristics.

The term “hydrophilic” and “hydrophilicity” refer to an affinity for water, while the terms “hydrophobic” and “hydrophobicity” refer to a lack of affinity for water. Hydrophobic materials typically correspond to those materials to which water has little or no tendency to adhere. As such, water on a surface of a hydrophobic material tends to bead up. One measure of hydrophobicity of a material is a contact angle between a surface of the material and a line tangent to a drop of water at a point of contact with the surface. Typically, the material is considered to be hydrophobic if the contact angle is greater than 90.degrees.

The term “electrically conductive” and “electrical conductivity” refer to an ability to transport an electric current. Electrically conductive materials typically correspond to those materials that exhibit little or no opposition to flow of an electric current.

The term “microstructure” refers to a microscopic structure of a material and can encompass, for example, a lattice structure, crystallinity, dislocations, grain boundaries, constituent atoms, doping level, surface functionalization, and the like. One example of a microstructure is an elongated structure, such as comprising a nanostructure. Another example of a microstructure is an array or arrangement of nanostructures.

The term “nanotube” refers to an elongated structure. Typically, a nanotube is substantially hollow and, thus, can exhibit characteristics that differ from those of certain elongated, solid structures. In some instances, a nanotube can be represented as comprising a cylindrical shape. A nanotube typically has a cross-sectional diameter from about 0.5 nanometer (“nm”) to about 1,000 nm, such as from about 1 nm to about 200 nm, from about 1 nm to about 100 nm, or from about 1 nm to about 50 nm, and a length from about 0.1 micrometer (“μm”) to about 1,000 μm, such as from about 1 μm to about 50 μm or from about 1 μm to about 10 μm. The terms “nanotube” and “nanostructure” are used interchangeably throughout.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. GENERAL

The transition from flagellar rod polymerization to hook polymerization is concurrent with the formation of bushings in the peptidoglycan (P-ring) and LPS (L-ring) layers, and outer-membrane penetration by the growing flagellar organelle. A stop-polymerization mechanism senses completion of the rod or drive-shaft component of the flagellar motor to allow the switch to polymerization of the next structure, the hook, which is a flexible coupling between the rod and external filament. When the rod intrinsic polymerization-termination signal was disrupted by mutation, several steps in flagellar assembly were affected: (1) the rods were elongated (polyrods) and in some cases hooks did not form, resulting in rod-filament structures; (2) the polyrod-length became controlled by the hook-length control protein, FliK; (3) the flagellum failed to penetrate the outer membrane and grew between the inner and outer membranes; (4) multiple P-rings formed on the polyrod structures; and (5) L-rings failed to form. FliK was shown to be a molecular tape measure; insertions and deletions in FliK result in correspondingly longer and shorter hooks or rods depending on the mutant background.

Bacteria swim, propelled by some of the tiniest, complex molecular motors in the biosphere (Kojima and Blair, 2004). A signal transduction cascade that comprises the bacteria's brain-like network controls flagellar rotation resulting in the movement of the bacterium up chemical gradients (Armitage et al., 2005). From a structural perspective, dozens of proteins have to come together by an ordered assembly process, many with intrinsic self-assembly properties to complete these molecular nano-machines built for movement (Minamino and Namba, 2004). Genetic mechanisms coordinate the regulation of gene expression to the assembly process (Aldridge and Hughes, 2002).

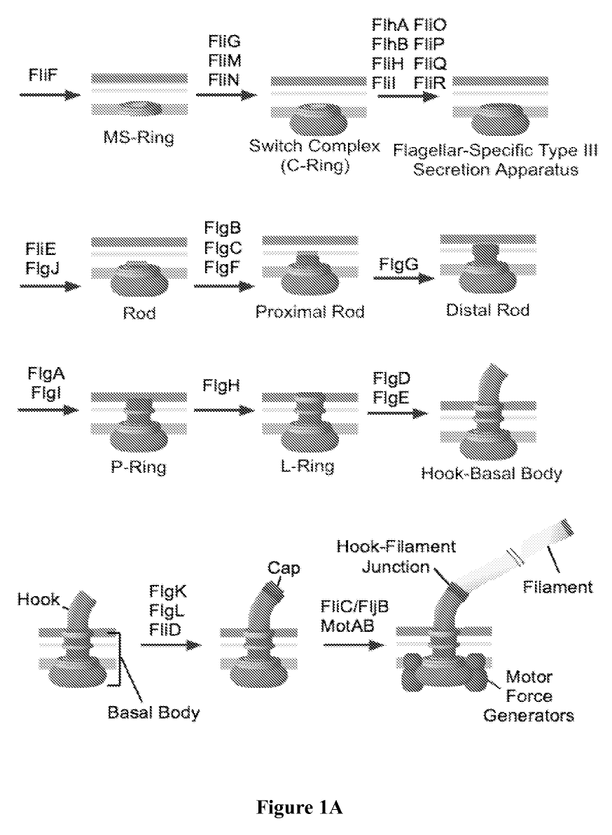

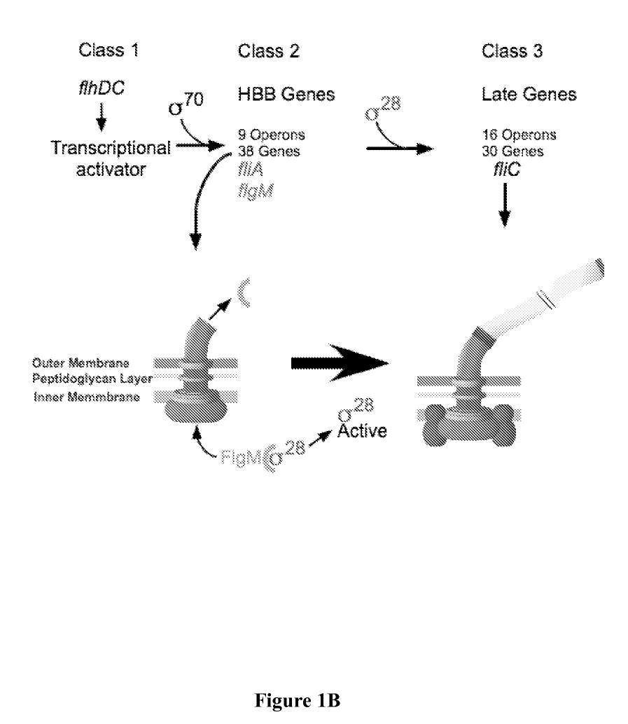

The bacterial flagellum is composed of three main substructures: the basal body (which functions as a transmembrane rotary motor), the hook (which serves as a universal joint permitting articulation between the motor and the filament), and the filament (the propeller) (Berg and Anderson, 1973; Macnab, 1996). Motor force generators assemble at the flagellum and utilize the energy of the proton motive force to drive the rotation of the flagellum resulting in propulsion. A flagellar-specific type III secretion (T3S) system transports precursor proteins through the hollow center of the structure during assembly (Macnab, 2004). Following secretion, protein subunits travel to the tip of the elongating structure where they assemble into place (Emerson et al., 1970; Iino, 1969). Flagellar assembly follows an ordered pathway of subunit addition (FIG. 1A; reviewed in (Aizawa, 1996)). Insertion of a ringed structure, the MS-ring, which is composed of subunits of a single protein into the inner membrane, is the initial step of basal body assembly. This is followed by the addition of the switch complex that interacts with the chemosensory system to control the direction of flagellar rotation and the flagellar-specific T3S apparatus, which is assembled into a cytoplasmic membrane disc within the MS-ring (Macnab, 1999). A drive-shaft (rod) structure extends from the MS-ring through the peptidoglycan layer. The rod assembles beneath the rod-scaffold, which is also a muramidase that digests the peptidoglycan layer as the rod polymerizes through it (Hirano et al., 2001). At this point in assembly, hook initiation can begin, but elongation outside the cell requires that the P- and L-rings be assembled first and replacement of the rod scaffold with the hook scaffold (Kubori et al., 1992; Ohnishi et al., 1994). The P- and L-rings act as bushings around the rod in the peptidoglycan and lipopolysaccharide (LPS) layers respectively. Addition of the L-ring effectively opens a hole in the LPS layer for the hook elongation to continue outside the cell. Upon hook completion, the flagellar T3S apparatus changes in specificity from hook-basal body (HBB) substrates to late assembly substrates to initiate assembly of the long external filament (Hirano et al., 2003; Makishima et al., 2001; Minamino et al., 1999a; Minamino and Macnab, 1999). The filament is assembled beneath the final capping scaffold, which provides a nucleation site for self-polymerization of flagellin (Yonekura et al., 2000). Filament elongation slows down exponentially as it grows. This is presumably due to competition between the positive force of flagellin subunit export and hindered diffusion due to friction between the subunits and the narrow inner walls of the channel, which increases with filament length.

Flagellar gene regulation is coupled to the assembly pathway (FIG. 1B) (Aldridge and Hughes, 2002; Chilcott and Hughes, 2000). A negative regulatory protein, FlgM, coordinates the transition from HBB completion to initiation of filament transcription by the flagellar-specific transcription factor, σ²⁸ (Karlinsey et al., 2000b). FlgM is an anti-σ²⁸ factor that inhibits σ²⁸-dependent transcription from class 3 promoters prior to HBB completion (Ohnishi et al., 1992). Upon HBB completion, FlgM is secreted from the cell and σ²⁸ is free to transcribe the late assembly genes now needed (Hughes et al., 1993; Kutsukake, 1994). In this way genes whose products are assembled outside the cytoplasm in the final assembly stage (in particular the large external filament) are not transcribed until the HBB structure onto which these late subunits will be added is completed.

Polyrod and super-polyrod structures can be utilized as protein nanotubing, as the structures can be separated according to size. Unlike the hook, which is designed to be flexible, the rod is the rigid drive shaft of the flagellar motor. Alternatively, mutants that control cell body size can be employed to control the final length of the super-polyrod structures.

The bacterial flagellar motor is an example of finished bio-nanotechnology. This motor has the same power-to-weight ratio as an internal combustion engine, spins at up to 100,000 rpm and achieves near-perfect efficiency.

The shape of the filament of bacterial flagella can be precisely controlled because it depends on the amino acid sequence in the flagellin, as well as the temperature and pH of the solution. For instance, the filaments can be changed from achiral rods to highly twisted helices that look like springs. Using a polarisation microscope, it has been found that helical filaments undergo a phase transition to a novel liquid crystalline state in which the flagella become cone-shaped for concentrations above a certain level. In contrast, this phase transition is not seen in experiments with rod-shaped filaments.

Nanostructures are of great interest not only for their basic scientific richness, but also because they have the potential to revolutionize critical technologies. The miniaturization of electronic devices over the past century has profoundly affected human communication, computation, manufacturing and transportation systems. True molecular-scale devices and structures are now emerging that set the stage for integrated nanoelectronics.

Nanotubes can serve as important nanoelectromechanical systems (NEMS)-enabling materials, since nanotubes can be engineered and modified to be part of a higher order system, i.e. as active components in a movable device. Cumings et al. (US 2002/0070426 Al, herein incorporated by reference in its entirety for its teachings concerning nanotubes) discloses a method for forming a telescoped multiwall carbon nanotube (“MWNT”). Such a telescoped multiwall nanotube can act as a linear bearing in an electromechanical system. That is, the walls of a multiwalled carbon nanotube are concentrically separated and are shown to telescope axially inwardly and outwardly. In Science 289:602 604 (28 Jul. 2000), a scientific publication related to the 2002/0070426 A1 patent publication, Cumings and Zettl describe a low friction nanoscale linear bearing, which operates in a reciprocal (i.e. telescoping) manner.

Den et al. U.S. Pat. No. 6,628,053 (herein incorporated by reference in its entirety for its teachings concerning nanotubes) discloses a carbon nanotube device comprising a support having a conductive surface and a carbon nanotube, wherein one terminus of the nanotube binds to the conductive surface so that conduction between the surface and the carbon nanotube is maintained. The device is used as an electron generator.

Falvo et al. Nature 397:236 238 (Jan. 21, 1997) (herein incorporated by reference in its entirety for its teachings concerning nanotubes) disclose studies involving the rolling of carbon nanotubes using atomic force microscope (AFM) manipulation of multiwall carbon nanotubes (MWCNT, termed in the paper “CNT”).

Minett et al. Current Applied Physics 2:61 64 (2002) (herein incorporated by reference in its entirety for its teachings concerning nanotubes) disclose the use of carbon nanotubes as actuators in which the driving force is obtained from a deformation of the nanotube when a charge is applied. The authors, in their review also disclose the preparation of a suspended carbon nanotube across two metallic contacts growth of nanotubes across two metal contacts in a process that involved E-beam lithography and selective patterning.

Cumings et al. Nature 406:586 (Aug. 10, 2000) (herein incorporated by reference in its entirety for its teachings concerning nanotubes) disclose techniques for peeling and sharpening multiwall nanotubes. These sharpened tubes are disclosed as having utility as biological electrodes, microscopic tips, etc.

Fraysse at al. Carbon 40:1735 1739 (2002) (herein incorporated by reference in its entirety for its teachings concerning nanotubes) discloses carbon nanotubes that act like actuators. A SWNT can be disposed above a substrate and between a pair of metal-on-oxide layers. The nanotubes act as actuators through a cantilever effect achieved through longitudinal deformation of the nanotube.

The nanotubes produced herein may be used in a variety of applications. For example, they can be used as reinforcements in fiber-reinforced composite structures or hybrid composite structures (i.e. composites containing reinforcements such as continuous fibers in addition to nanotubes). The composites may further contain fillers such as carbon black, silica, and mixtures thereof. Examples of reinforceable matrix materials include inorganic and organic polymers, ceramics (e.g., Portland cement), carbon, and metals (e.g., lead or copper). When the matrix is an organic polymer, it may be a thermoset resin such as epoxy, bismaleimide, polyimide, or polyester resin; a thermoplastic resin; or a reaction injection molded resin. The nanotubes can also be used to reinforce continuous fibers. Examples of continuous fibers that can be reinforced or included in hybrid composites are aramid, carbon, glass fibers, and mixtures thereof. The continuous fibers can be woven, knit, crimped, or straight.

Tubes or hollow fibers having small internal diameters are known and are employed, in particular, for separation purposes, for example in medical dialysis, for gas separation or osmosis of aqueous systems, for example for water treatment (see Kirk-Othmer, Encyclopedia of Chemical Technology, 4.sup.th Ed., Vol. 13, pp. 312-313). The fiber material usually consists of polymers, which may in addition have pores, i.e. properties of semi-permeable membranes.

Hollow fibers with or without a core can be used, in particular, as separation or storage medium for gases, liquids or particle suspensions and for the filtration or purification of substance mixtures. Possible uses include membranes for gases, in particular H₂ or liquids, for particle filtration, in chromatography, for oil/water separation, as ion exchangers in dialysis, for size separation of cells, bacteria or viruses, as a constituent of an artificial lung, for desalination for water removal or irrigation or as a filter for dewatering of fuels.

Hollow fibers can furthermore be used in sensor technology for solvent, gas, moisture or biosensors, in capillary electrophoresis, in catalytic systems, in scanning probe microscopy or as materials in superlight construction, as mechanical reinforcement analogously to glass fibers, as sound or vibration protection as a composite material or filler, as a controlled release or drug delivery system, in medical separation methods, in dialysis, as an artificial lung, protein store or in tissue engineering.

In the clothing/textiles industry, the hollow fibers according to the invention can be used as thermal insulator in clothing or sleeping bags, in photochromic or thermochromic clothing through embedding of dyes in the interior of the tubes or as labels through markers in the interior of the tubes.

Hollow fibers are also used in electronics, optics or energy recovery. For example, the hollow fibers can be used for the production of wires, cables or capacitors, micromachines (for example for piezoelectric shaping, nanoperistaltic pumps or for the shaping of photoaddressable polymers) or interlayer dielectrics. Further uses of hollow fibers according to the invention are microreactors, for example for catalytic reactors, template reactions and bioreactors, heat generation through conversion of sunlight (solar .alpha. systems), in chip technology as flexible devices or microscopy as a sensor constituent (for example as tips or probes for scanning probe microscopes or SNOM instruments).

Hollow fibers can be incorporated or introduced into cell membranes and used for the separation and recovery or removal of metabolites, enzymes and other components of the cytoplasm within cells or cytoplasmitic components and thus for the recovery of bio-pharmaceuticals. They can also be used as flagella to motorize various cells.

### C. COMPOSITIONS

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular protein is disclosed and discussed and a number of modifications that can be made to a number of molecules are discussed, specifically contemplated is each and every combination and permutation of the protein and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

1. Nucleic acids

There are a variety of molecules disclosed herein that are nucleic acid based, including for example the nucleic acids that encode, for example, FlgG and FliK as well as any other proteins disclosed herein, as well as various functional nucleic acids. The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantagous that the antisense molecule be made up of nucleotide analogs that reduce the degradation of the antisense molecule in the cellular environment.

a) Nucleotides and related molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate).

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989, 86, 6553-6556),

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

b) Sequences

There are a variety of sequences related to, for example, FlgG and FliK as well as any other protein disclosed herein that are disclosed on Genbank, and these sequences and others are herein incorporated by reference in their entireties as well as for individual subsequences contained therein. It is understood that those given sequences have homology to other organisms, and by relating an example of a protein or nucleic acid from one organism (such as SEQ ID NO: 1 from Salmonella enterica serovar Typhimurium strain LT2) disclosed herein are also those sequences from other organisms which have homology to that given organism.

A variety of sequences are provided herein and these and others can be found in Genbank, at www.pubmed.gov. Those of skill in the art understand how to resolve sequence discrepancies and differences and to adjust the compositions and methods relating to a particular sequence to other related sequences.

2. Nucleic Acid Delivery

In the methods described above which include the administration and uptake of exogenous DNA into a cell (i.e., gene transduction or transfection), the disclosed nucleic acids can be in the form of naked DNA or RNA, or the nucleic acids can be in a vector for delivering the nucleic acids to the cells, whereby the DNA fragment is under the transcriptional regulation of a promoter, as would be well understood by one of ordinary skill in the art. The vector can be a commercially available preparation, such as an adenovirus vector (Quantum Biotechnologies, Inc. (Laval, Quebec, Canada). Delivery of the nucleic acid or vector to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art.

As one example, vector delivery can be via a viral system, such as a retroviral vector system which can package a recombinant retroviral genome (see e.g., Pastan et al., Proc. Natl. Acad. Sci. U.S.A. 85:4486, 1988; Miller et al., Mol. Cell. Biol. 6:2895, 1986). The recombinant retrovirus can then be used to infect and thereby deliver to the infected cells nucleic acid encoding a broadly neutralizing antibody (or active fragment thereof). The exact method of introducing the altered nucleic acid into mammalian cells is, of course, not limited to the use of retroviral vectors. Other techniques are widely available for this procedure including the use of adenoviral vectors (Mitani et al., Hum. Gene Ther. 5:941-948, 1994), adeno-associated viral (AAV) vectors (Goodman et al., Blood 84:1492-1500, 1994), lentiviral vectors (Naidini et al., Science 272:263-267, 1996), pseudotyped retroviral vectors (Agrawal et al., Exper. Hematol. 24:738-747, 1996). Physical transduction techniques can also be used, such as liposome delivery and receptor-mediated and other endocytosis mechanisms (see, for example, Schwartzenberger et al., Blood 87:472-478, 1996). This disclosed compositions and methods can be used in conjunction with any of these or other commonly used gene transfer methods.

3. Delivery of the Compositions to Cells

There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991) Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

a) Nucleic Acid Based Delivery Systems

Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)).

As used herein, plasmid or viral vectors are agents that transport the disclosed nucleic acids, such as FlgG into the cell without degradation and include a promoter yielding expression of the gene in the cells into which it is delivered. In some embodiments the vectors are derived from either a virus or a retrovirus. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors can have higher transaction (ability to introduce genes) abilities than chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

(1) Retroviral Vectors

A retrovirus is an animal virus belonging to the virus family of Retroviridae, including any types, subfamilies, genus, or tropisms. Retroviral vectors, in general, are described by Verma, I. M., Retroviral vectors for gene transfer. In Microbiology-1985, American Society for Microbiology, pp. 229-232, Washington, (1985), which is incorporated by reference herein. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

(2) Adenoviral Vectors

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

A viral vector can be one based on an adenovirus which has had the El gene removed and these virons are generated in a cell line such as the human 293 cell line. In another preferred embodiment both the E1 and E3 genes are removed from the adenovirus genome.

(3) Adeno-Associated Viral Vectors

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, Calif., which can contain the herpes simplex virus thymidine kinase gene, HSV-tk, and/or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus.

Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference for material related to the AAV vector.

The disclosed vectors thus provide DNA molecules which are capable of integration into a mammalian chromosome without substantial toxicity.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

(4) Large Payload Viral Vectors

Molecular genetic experiments with large human herpesviruses have provided a means whereby large heterologous DNA fragments can be cloned, propagated and established in cells permissive for infection with herpesviruses (Sun et al., Nature genetics 8: 33-41, 1994; Cotter and Robertson, Curr Opin Mol Ther 5: 633-644, 1999). These large DNA viruses (herpes simplex virus (HSV) and Epstein-Barr virus (EBV), have the potential to deliver fragments of human heterologous DNA >150 kb to specific cells. EBV recombinants can maintain large pieces of DNA in the infected B-cells as episomal DNA. Individual clones carried human genomic inserts up to 330 kb appeared genetically stable The maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA>220 kb and to infect cells that can stably maintain DNA as episomes.

Other useful systems include, for example, replicating and host-restricted non-replicating vaccinia virus vectors.

b) Non-Nucleic Acid Based Systems

The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

73. Thus, the compositions can comprise, in addition to the disclosed vectors for example, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J. Resp. Cell. Mol. Biol. 1:95-100 (1989); Felgner et al. Proc. Natl. Acad. Sci. USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis., as well as other liposomes developed according to procedures standard in the art. In addition, the disclosed nucleic acid or vector can be delivered in vivo by electroporation, the technology for which is available from Genetronics, Inc. (San Diego, Calif.) as well as by means of a SONOPORATION machine (InaRx Pharmaceutical Corp., Tucson, Ariz.).

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). These techniques can be used for a variety of other specific cell types. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

Nucleic acids that are delivered to cells which are to be integrated into the host cell genome, typically contain integration sequences. These sequences are often viral related sequences, particularly when viral based systems are used. These viral integration systems can also be incorporated into nucleic acids which are to be delivered using a non-nucleic acid based system of deliver, such as a liposome, so that the nucleic acid contained in the delivery system can be come integrated into the host genome.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

4. Expression Systems

The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

a) Viral Promoters and Enhancers

Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell. Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell. Bio. 4:1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, -fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promoter and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTR.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

b) Markers

The viral vectors can include nucleic acid sequence encoding a marker product. This marker product is used to determine if the gene has been delivered to the cell and once delivered is being expressed. Preferred marker genes are the E. Coli lacZ gene, which encodes β-galactosidase, and green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

5. Peptides

a) Protein Variants

As discussed herein there are numerous variants of the FlgG and FliK protein that are known and herein contemplated. In addition, to the known functional FlgG and FliK strain variants there are derivatives and mutants of these proteins which can be used with the disclosed methods and compositions. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

TABLE 2
Amino Acid Substitutions
Original Residue Exemplary Conservative
Substitutions, others are known in the art.

Ala; ser
Arg; lys, gln
Asn; gln; his
Asp; glu
Cys; ser
Gln; asn, lys
Glu; asp
Gly; pro
His; asn; gln
Ile; leu; val
Leu; ile; val
Lys; arg; gln
Met; leu; ile
Phe; met; leu; tyr
Ser; thr
Thr; ser
Trp; tyr
Tyr; trp; phe
Val; ile; leu

Substantial changes in function or immunological identity are made by selecting substitutions that are less conservative than those in Table 1, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W.H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. For example, SEQ ID NO: 1 sets forth a particular sequence of FlgG. Specifically disclosed are variants of these and other proteins herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. It is also understood that while no amino acid sequence indicates what particular DNA sequence encodes that protein within an organism, where particular variants of a disclosed protein are disclosed herein, the known nucleic acid sequence that encodes that protein in the particular sequence from which that protein arises is also known and herein disclosed and described.

It is understood that there are numerous amino acid and peptide analogs which can be incorporated into the disclosed compositions. For example, there are numerous D amino acids or amino acids which have a different functional substituent then the amino acids shown in Table 1. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way (Thorson et al., Methods in Molec. Biol. 77:43-73 (1991), Zoller, Current Opinion in Biotechnology, 3:348-354 (1992); Tbba, Biotechnology & Genetic Engineering Reviews 13:197-216 (1995), Cahill et al., TIBS, 14(10):400-403 (1989); Benner, TIB Tech, 12:158-163 (1994); Ibba and Hennecke, Bio/technology, 12:678-682 (1994) all of which are herein incorporated by reference at least for material related to amino acid analogs).

Molecules can be produced that resemble peptides, but which are not connected via a natural peptide linkage. For example, linkages for amino acids or amino acid analogs can include CH₂NH—, —CH₂S—, —CH₂—CH₂—, —CH═CH—(cis and trans), —COCH₂—, —CH(OH)CH₂—, and —CHH₂SO—(These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CH H₂—S); Hann J. Chem. Soc Perkin Trans. I 307-314 (1982) (—CH—CH—, cis and trans); Ahnquist et al. J. Med. Chem. 23:1392-1398 (1980) (—COCH₂—); Jennings-White et al. Tetrahedron Lett 23:2533 (1982) (—COCH₂—); Szelke et al. European Appln, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations. (Rizo and Gierasch Ann. Rev. Biochem. 61:387 (1992), incorporated herein by reference).

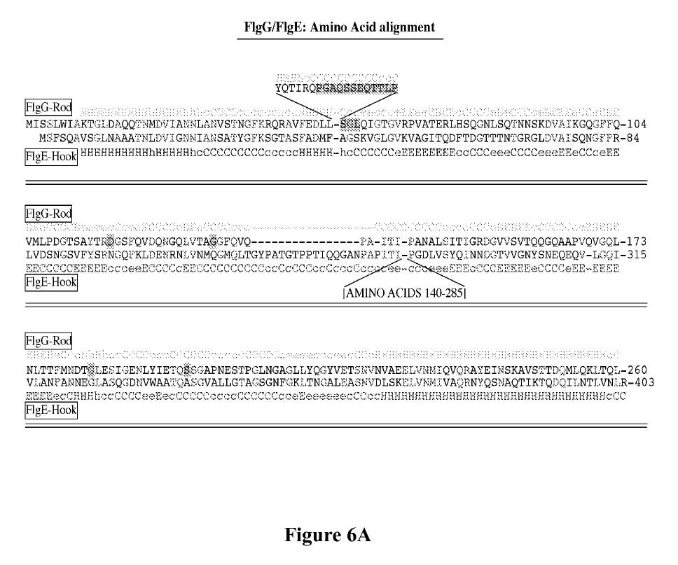

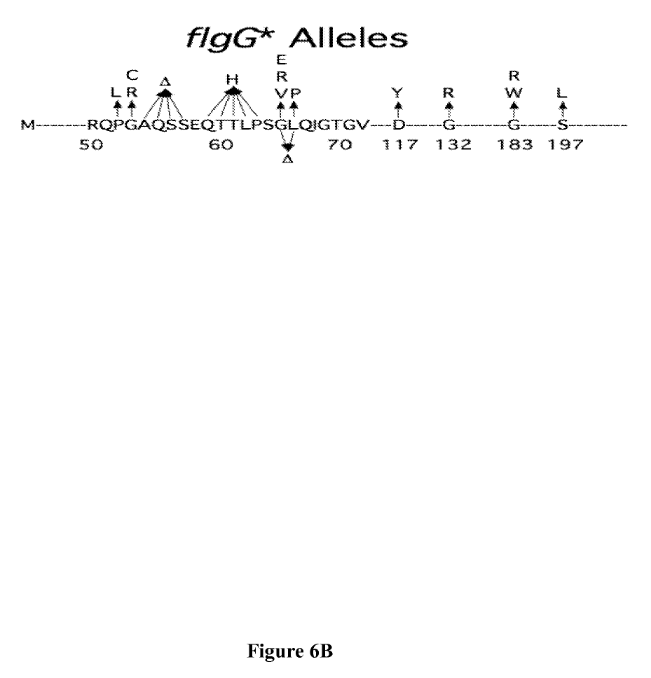

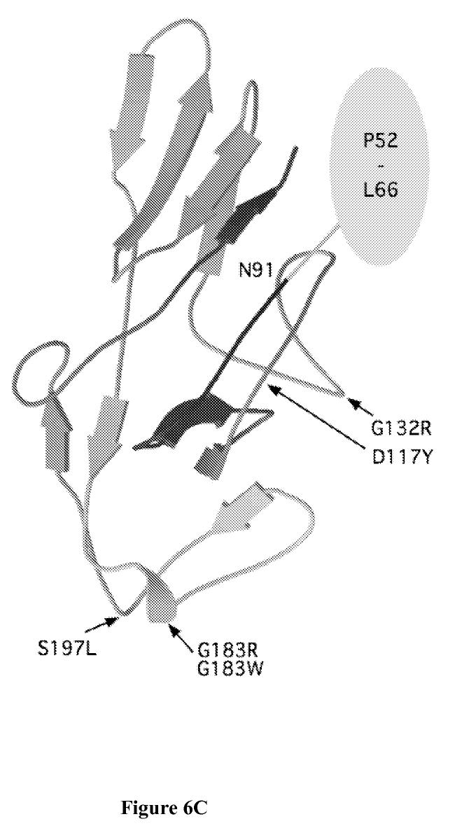

Disclosed herein is a composition comprising an engineered bacterial flagella that is greater in length than a flagella from a naturally occurring organism. The engineered flagella can be 10%, 20%, 30%, 40%, 50%, 60%, 70%, 80%, 90%, 100%, 1.5 times, 2 times, 3 times, 4 times, 5 times, 6 times, 7 times, 8 times, 9 times, or 10 or more times longer than that of a naturally occurring organism. For example, the engineered flagella can be greater than 0.1, 0.2, 0.3, 0.4, 0.5, 0.6, 0.7, 0.8, 0.9, 1.0, 1.5, 2.0, 2.5, 3.0, 3.5, 4.0, 4.5, or 5.0 μm or more. In one example, the engineered flagella can form a helix. The engineered bacterial flagella can also comprise a mutation in FlgG protein. In one example, the mutation in the FlgG protein can be found in amino acid residues 52-68 of SEQ ID NO: 1. Two polyrod mutant sites that include the G183R/G183W and S197L mutations, reside close to each other at the very bottom of the predicted FlgG structural model, and two other polyrod mutant sites that include the D117Y and R132G mutations, are located close to each other in the middle of the structure (FIG. 6C). This allowed for the discovery of the mechanisms for FlgG stop-polymerization. That is, the 52-68 amino acid region and the amino acids at the bottom of the FlgG structure G183 and S197 interact to stop FlgG polymerization. This stacking of FlgG subunits likely results in a conformational change mediated through the region containing the D117 and R132 residues that prevents further FlgG polymerization. In this example, the mutation can be a single amino acid mutation, such as a substitution, deletion, or insertion. The mutation can also comprise more than one amino acid residue. The engineered bacterial flagella can comprise a polyrod, such as a nanotube.

In one example, the nanotube can also comprise a binding agent having an affinity for a biomolecule of interest coupled to the surface of the nanotube. Examples are given in US Patent Application 20070092870, herein incorporated by reference in its entirety for its teaching concerning binding agents on nanotubes. Use of the phrase “biomolecule” is intended to encompass deoxyribonucleic acid (DNA), ribonucleic acid (RNA), nucleotides, oligonucleotides, nucleosides, proteins, peptides, amino acids, polypeptides, selenoproteins, antibodies, antigens, protein complexes, viruses and other molecular pathogens and toxins, combinations thereof, and the like. In particular, the biomolecule can include, but is not limited to, naturally occurring substances such as polypeptides, polynucleotides, lipids, fatty acids, glycoproteins, carbohydrates, fatty acids, fatty esters, macromolecular polypeptide complexes, vitamins, co-factors, microorganisms such as viruses, bacteria, protozoa, archaea, fungi, algae, spores, apicomplexan, trematodes, nematodes, mycoplasma, or combinations thereof, as well as cells (e.g., eukaryotic cells and prokaryotic cells) infected with viruses, toxins, and/or other molecular pathogens.

The biomolecule can be a virus, including, but not limited to, RNA and DNA viruses. In particular the biomolecule is a virus, which may include, but is not limited to, a retrovirus (e.g., human immunodeficiency virus (HIV), a feline immunodeficiency virus (FIV), a simian immunodeficiency virus (SIV), a porcine immunodeficiency virus (PIV), a feline leukemia virus, a bovine immunodeficiency virus, a bovine leukemia virus, a equine infectious anemia virus, a human T-cell leukemia virus), a Pneumovirus (e.g., respiratory syncytial virus (RSV)), Paramyxoviridae (e.g., Paramyxovirus (Parainfluenzavirus 1-4, Sendai virus, mumps, Newcastle disease virus)), a Metapneumovirus (e.g., human and avian metapneumovirus), and Orthomyxoviridae (e.g., an influenza virus A, B, C). In addition, the biomolecule may include additional viruses including, but not limited to, an astrovirideae, a calivirideae, a herpes virus, a picornaviridea, a poxuvirideae, a reovirideae, a togavirideae, an avian influenza virus, a polyomavirus, an adenovirus, a rhinovirus, a Bunyavirus, a Lassa fever virus, an Ebola virus, a corona virus, an arenavirus, a Filovirus, a rhabdovirus, an alphavirus, a flavivirus, Epstein-Barr Virus (EBV), and viruses of agricultural relevance such as the Tomato Spotted Wilt Virus.

In another exemplary embodiment, the biomolecule is a surface molecule or surface antigen on the surface of a pathogen (e.g., a bacterial cell, a spore, etc.), or the biomolecule is a toxin or other byproduct of a pathogen (e.g., a toxin produced by a bacterial cell). Other examples of biomolecules are viral projections such as Hemmaglutinin and Neuraminidase.

Use of the term “affinity” can include biological interactions and/or chemical interactions. The biological interactions can include, but are not limited to, bonding or hybridization among one or more biological functional groups located on the binding agent and the biomolecule of interest. In this regard, the binding agent can include one or more biological functional groups that selectively interact with one or more biological functional groups of the biomolecule of interest. The chemical interaction can include, but is not limited to, bonding (e.g., covalent bonding, ionic bonding, and the like) among one or more functional groups (e.g., organic and/or inorganic functional groups) located on the biomolecule of interest and binding agent. The nanotube can further comprise a reporter molecule coupled to the surface of the nanotube, wherein the reporter molecule is capable of providing a signal.

The biomolecule of interest is selected from at least one of the following: a polypeptide, a protein, a glycoprotein, a nucleic acid, a carbohydrate, a lipid, a vitamin, a virus, a virus infected cell, and combinations thereof. The biomolecule can comprise an RNA or DNA virus. The binding agent can be selected from one of the following: a metal, a polymer, a plastic, a polynucleotide, a polypeptide, a protein, an amino acid, a glycoprotein, a lipid, a carbohydrate, a fatty acid, a fatty ester, a macromolecular polypeptide complex, and a combination thereof. In one example, the amino acid can comprise histidine.

Also disclosed herein is a vector comprising flgG, wherein flgG comprises a mutation that allows for the formation of a polyrod. The mutation can comprise a mutation in one or more amino acid residues 52-68 of SEQ ID NO: 1. The vector can further comprise a mutation in fliK, as disclosed in Example 1.

Further disclosed is a cell expressing the vector disclosed herein. Said cell can be in a system, or in an organism. For example, disclosed herein is a transgenic organism that produces a bacterial flagella greater in length than a naturally occurring flagella. The organism can be a bacterium, such as Salmonella.

### D. METHODS

Disclosed herein is a method of producing a polyrod, comprising: a) introducing into a cell a vector as disclosed herein; b) expressing the polypeptide encoded by the vector of step a); thereby producing a polyrod. The cell can also express other proteins needed for producing flagella. Examples can be found in Example 1. The polyrod can be used as a nanotube. The nanotube can also comprise a binding agent having an affinity for a biomolecule of interest coupled to the surface of the nanotube, as described above. The binding agent can be histidine, and these histidine residues can produced by inserting his codons into the vector. Such methods are known to those skilled in the art.

Also disclosed herein are methods of sequencing. Such methods are disclosed in US Patent application 20060086626, herein incorporated by reference in its entirety for its teaching concerning sequencing using nanotubes.

It has been demonstrated that a voltage gradient can drive single stranded polynucleotides through a nanometer diameter transmembrane channel, or nanotube. Kasianowicz, J. J. et al., Proc. Natl. Acad. Sci. USA 93, 13770-13773 (1996). During the translocation process, the extended polynucleotide molecules will block a substantial portion of the otherwise open nanotube channel. This blockage leads to a decrease in the ionic current flow of the buffer solution through the nanopore during the polynucleotide translocation. By measuring the magnitude of the reduced ionic current flow during translocation, the passage of a single polynucleotide can be monitored by recording the translocation duration and blockage current, yielding plots with characteristic sensing patterns. By controlling translocation conditions, the lengths of individual polynucleotide molecules can be determined from the calibrated translocation time. In addition, the differing physical and chemical properties of the individual bases comprising the polynucleotide strand generate a measurable and reproducible modulation of the blockage current that allows an identification of the specific base sequence of the translocating polynucleotide. Kasianowicz, J. J. et al., Proc. Natl. Acad. Sci. USA 93, 13770-13773 (1996); Akeson, M. et al., Biophys. J. 77, 3227-3233 (1999).

Another means of detecting a polynucleotide translocating a nanotube is based on quantum mechanical tunneling currents through the proximal base of the translocating strand as it passes between a pair of metal electrodes placed adjacent to the nanopore on the same surface of the underlying substrate. Measuring the magnitude of the tunneling current would be an electronic method for detecting the presence of a translocating molecule, and if the conditions were adequately controlled and the measurements sufficiently sensitive, the sequence of constituent bases could be determined. One of the primary motivations for this approach is that typical tunneling currents in scanning tunneling microscopes are on the order of 1-10 nanoamps. This is two to three orders of magnitude larger than the ionic currents observed during polymer translocation of 2 nanometer nanopores.

### E. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

### Length Determination of the Bacterial Flagellar Motor Drive Shaft is Intrinsic to the Rod Structural Subunit FlgG and Hook-Length Control Protein FliK

a) Isolation of flagellar proximal rod (flgG) alleles by selection for Flk-bypass mutants

In strains that are defective in the formation of the HBB, FlgM is not secreted and accumulates to inhibit σ²⁸-dependent transcription from class 3 promoters. Null alleles of flgM restore σ²⁸-dependent transcription in HBB mutants (Gillen and Hughes, 1991a; Gillen and Hughes, 1991b). In HBB mutants with specific defects in P- or L-ring formation, null alleles in either the flk or flgM loci restore σ²⁸-dependent transcription (Gillen and Hughes, 1991a; Karlinsey et al., 1997). For clarity, the Flk protein will be referred to as “Fluke” to avoid confusion with the FliK protein discussed later. Fluke prevents premature secretion of FlgM into the periplasm in strains missing the P- or L-rings (ΔflgHI) (Aldridge et al., 2006). Loss of Fluke allows FlgM secretion into the periplasm where it is degraded in P- and L-ring mutants. Loss of Fluke does not restore σ²⁸-dependent transcription in mutants defective in assembly steps before rod completion, such as an flhA mutant (an integral membrane component of the flagellar secretion system). Thus, a flk null can be distinguished from a flgM null in a ΔflgHI (deleted for P- and L-ring structural genes) flhA^{ts} (temperature-sensitive) strain. At 30° C. (FlgHI⁻ FlhA⁺) either loss of FlgM or loss of Fluke (by allowing FlgM secretion into the periplasm) allows σ²⁸-dependent transcription. At 42° C. (FlgHI⁻ FlhA⁻), only loss of FlgM allows σ²⁸-dependent transcription. Since there is no rod structure assembled without FlhA, then loss of Fluke does not result in FlgM secretion into the periplasm because it has no mechanism for secretion. In a screen for Fluke-bypass mutants (Fluke^{BP}), which are mutants phenotypically identical to Fluke null mutants, but remain Fluke⁺, a rare class of mutants was isolated that mapped to the HBB structural operon, the flg operon (see Experimental Procedures). Fine mapping, followed by DNA sequence analysis revealed that the majority of Fluke^{BP} mutants were in the structural gene coding for the distal rod subunit, FlgG (Okino et al., 1989). The initial set of Fluke^{BP} mutants located to the flgG gene resulted from short deletions and substitution mutations that exhibited the Fluke^{BP} phenotype (increased σ²⁸-dependent transcription in strains defective for the P- or L-ring structural genes).

b) Fluke-Bypass Alleles in flgG Form Flagellar Rods of Unusual Size (Polyrod Structures)

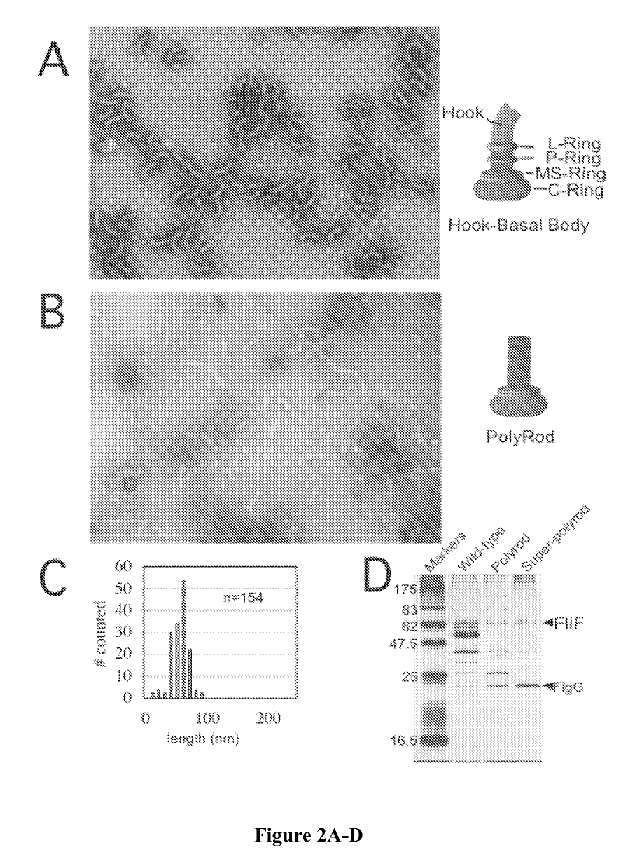

Mutants were found in the rod structural gene flgG that would overcome FlgM inhibition of σ28-dependent transcription in HBB mutants specific to P- or L-ring formation. Because FlgG is a rod structural gene, the mutants were examined by electron microscopy to determine if the flgG regulatory mutants (flgG*) affected HBB formation in any way. Basal structures were examined from a wild-type strain (FIG. 2A) and from a flgG regulatory mutant strain (FIG. 2B). The flgG regulatory mutants resulted in structures that have never been reported: rods of unusual size (ROUS). FlgG-mutant rod lengths were compared to the lengths of normal rods from a flgG⁺ strain and were found to be about three times (average of 60 nm) the length of wild-type rod structures (22 nm) and have been termed polyrods (FIG. 2C).

The flagellar rod is composed of four structural subunits FlgB, FlgC, FlgF and FlgG. The FlgB, FlgC and FlgF proteins make up the proximal rod and are attached to the flagellar T3S apparatus through interactions with FlGB (Minamino et al., 2000). The FlgG subunits make up the distal rod structure. Flagellar basal structures isolated from the flgG* mutants were analyzed and found to have excess FlgG protein while the amounts of the other rod components remained the same as in wild-type rod structures (FIG. 2D). This indicates that FlgG polymerization was affected in the polyrod mutants.

c) Polyrod Structures Result in the Flagellar Type III Secretion-Specificity Switch

How could a doubling of the rod length result in reduced FlgM inhibition of σ²⁸-dependent transcription in the ring mutant strains? FlgM stability was measured to determine if the flgG regulatory mutation resulted in increased FlgM turnover. It did. Mutants defective in HBB formation such as P- and L-ring mutants accumulate FlgM in the cell resulting in the inhibition of σ²⁸-dependent transcription. However, the flgG* mutant resulted in increased FlgM turnover. This was the exact phenotype observed in a ring-defective strain missing Fluke and explains why they were obtained as Fluke-bypass mutants (Aldridge et al., 2006).

Both flagellar ROUSs and increased FlgM turnover resulted from the flgG* mutations. The increased FlgM turnover would account for the reduction in FlgM inhibition of σ²⁸-dependent transcription in ring-defective strains. But how does increasing the length of the rod result in increased FlgM turnover? One possibility is that rod extension in the flgG* mutants resulted in the switch of the flagellar secretion apparatus to late substrate secretion specificity, which includes FlgM and filament. Secretion of FlgM outside the cell is not possible in a ring mutant strain, but export into the periplasm is. The ability of the polyrod structure to export FlgM into the periplasm was tested using a FlgM-β-lactamase (FlgM-Bla) fusion as a reporter for secretion of FlgM into the periplasm. Export of FlgM-Bla into the periplasm would confer ampicillin resistance (Ap^{R}) to the cell (Aldridge et al., 2006). Cells unable to export FlgM-Bla would remain Ap^{S}. In a HBB⁺ strain, FlgM-Bla is secreted from the cell and into the supernatant. In a ring-defective strain, FlgM-Bla is retained in the cytoplasm (Aldridge et al., 2006). In both cases, the cells are Ap^{S}, indicating the FlgM-Bla is not in the periplasm (Table 1). However, in the ring-defective strain, the presence of the flgG* mutation (polyrod) conferred Ap^{R} to the cells indicating export of FlgM-Bla into the periplasm (Table 1). This is consistent with the model that the increase in rod-length allowed export of FlgM in ring-defective strains where it entered the periplasm and was degraded. Fusion of Bla to the C-terminus stabilizes FlgM in the periplasm and confers ampicillin resistance (Aldridge et al., 2006).

Export of FlgM-Bla into the periplasm provided a positive selection for null mutants in Fluke and Flk^{BP} mutants. A strain (TH10406) was constructed that lacked the P- and L-ring structural genes (ΔflgHI), contained a flgM-bla fusion and had two copies of the Fluke gene (flk⁺), one was the wild-type locus and the second was expressed from the arabinose promoter P_{araBAD}::flk⁺). Ampicillin resistant mutants were selected in this background and screened for those that remained ampicillin resistant (Ap^{R}) in the presence of arabinose to screen against Fluke null mutants. DNA sequence analysis was performed on the flgG locus of twenty-one independent isolates that were Ap^{R} in the presence of arabinose. Thirteen had substitutions in FlgG of nine were identical to those obtained previously. These results confirmed that the Fluke-bypass mutants result in a change in the flagellar secretion substrate specificity switch to late secretion substrates. The remaining eight Ap^{R} alleles await future characterization.

d) The Hook-Length Control Protein, FliK, Determines Polyrod Length: Super-Polyrods

These data show that the flagellar secretion substrate specificity switch is not dependent on HBB completion. Upon completion of the HBB structure, FlgM secretion occurs and σ²⁸-dependent late flagellar gene transcription begins (Karlinsey et al., 2000b). The switch is dependent primarily on the interaction between two proteins FliK and FlhB. The FliK protein is believed to measure hook completion and to interact with FlhB, an integral membrane component of the flagellar T3S apparatus that is ultimately responsible for the switch from hook-type substrate secretion to late-substrate secretion (Minamino and Pugsley, 2005). In the absence of FliK, the switch does not occur and some of the hook structures continue to grow creating the polyhook phenotype (originally called superhook) (Patterson-Delafield et al., 1973). Mutants in FlhB have been isolated that allow late secretion in the absence of FliK supporting the model that FlhB is the protein responsible for secretion substrate specificity (Hirano et al., 1994; Kutsukake et al., 1994).

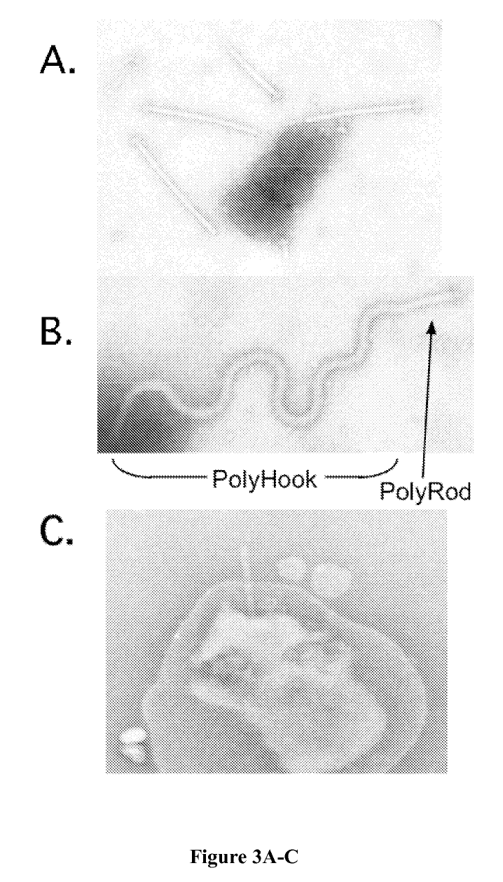

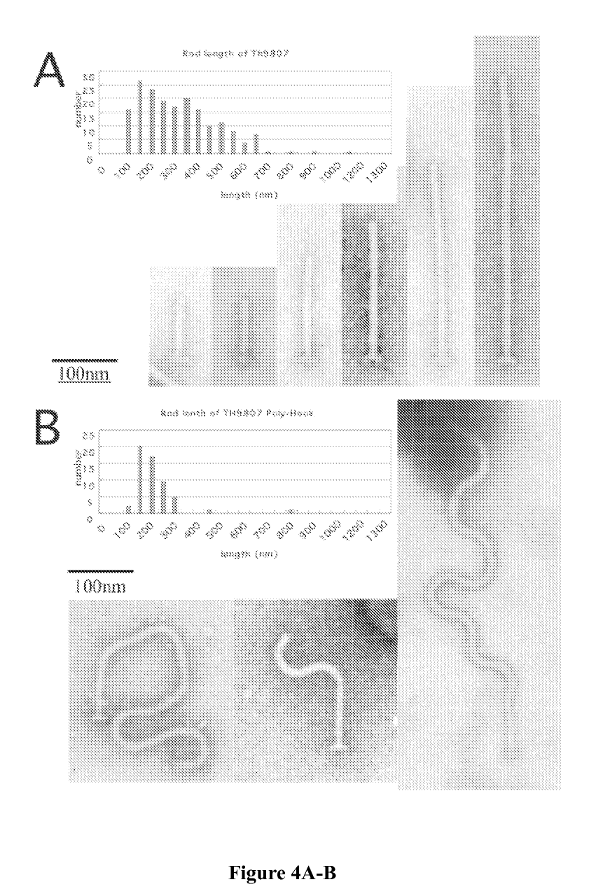

To test if FliK was required to make the switch allowing FlgM secretion into the periplasm in the ring-defective flgG* mutant background, a fliK null allele was introduced by P22 transduction into the flgG* strain that was also deleted for the P- and L-ring structural genes (ΔflgHI). Introduction of a fliK null allele (fliK::Tn10) restored FlgM-dependent inhibition of flagellar class 3 gene expression indicating that FlgM was no longer exported into the periplasm. This showed that FliK protein was still required for the switch allowing FlgM secretion into the periplasm. The basal structures of the flgG* ΔflgHI fliK::Tn10 strain were isolated and examined by electron microscopy and some remarkable structures were observed, super-polyrods that reached sizes up to 1,000 nm, which is the length of the cell (FIGS. 3 & 4). This shows that the upper limit to the growth of the super-polyrod is at least the size of the cell since the super-polyrod grows between the inner and outer membranes of the cell and are only visible outside the cell following osmotic shock treatment (FIGS. 3B & 3C). Other structures were observed where the transition from rod elongation to hook elongation occurred resulting in super-polyrod-polyhook structures (FIGS. 3B & 4B). Control of polyrod length by FliK was surprising because it was thought that FliK was specific to hook-length control. These data show that FliK is a measuring device for the final length of any rod-hook combination and not just hook-length. This was confirmed (see below).

e) FliK is a Molecular Measure for Flagellar Hook-Length Determination

In the Yersinia enterocolitica type III system, it was determined that the final length of the injectasome needle structure was determined by the FliK homolog, YscP. Insertions and deletions in YscP resulted in lengthening or shortening of the needle structure by about 0.17 nm per amino acid residue (Journet et al., 2003). This led to the proposal that YscP acts as a molecular ruler or tape measure that is alpha-helical in structure (0.15 nm per residue versus 0.4 nm per residue for unstructured polypeptide (Nelson and Cox, 2000)). The possibility the FliK could act as a molecular tape measure was investigated.

FliK is secreted through the flagellar basal structure prior to hook completion; the secretion signal is within the N-terminal 100 amino acid residues of the 405 residue FliK protein (Minamino et al., 1999b). A C-terminal domain of FliK within amino acids 204 through 370 interacts with the FlhB component of the flagellar type III secretion apparatus to flip the switch from rod-hook type secretion substrates to late secretion substrates, which includes the filament structural proteins FliC and FljB and the regulator of late flagellar gene transcription, FlgM (Minamino et al., 2004). Between the N-terminal secretion signal region and C-terminal secretion-specificity switch domain is a proline-rich region, which, based on analogy to the YscP molecular tape measure would contain the potential FliK tape measure region. In fact, when amino acids 147 through 266 was inserted into YscP, the needle structures increased by 20 nm indicating that this region of FliK can act as part of the alpha-helical tape measure (Journet et al., 2003).

A series of insertions of amino acids derived from the YscP proline-rich, tape-measure region were constructed in FliK and their effect of hook-length control was determined (Table 2). Positions in the N-terminal secretion signal between residues 50 & 51 and between residues 95 & 96 were chosen as well as positions in the proline-rich region between residues 140 & 141, residues 160 & 161, and residues 180 & 181. Remarkably, of fifteen YscP-sequence insertion mutants constructed in FliK, almost all insertions resulted in an increase in flagellar hook length of about 0.17 nm per residue. Only one, a 75 amino acid insertion between residues 50 and 51 of FliK, did not affect hook-length. Two insertions of amino acid residues from flagellar late secretion substrates FlgM and FliC were also constructed. A 75 amino acid insertion of FlgM residues between resides 140 and 141 of FliK increased hook length from 55+/−5 nm to 62.3+/−9.6 nm, while a 164 amino acid insertion of FliC sequence inserted at that same position increased hook-length to 84.6+/−21.5 nm. These lengths were similar to those observed with equivalent lengths of YscP insertion alleles.

To determine the effects of protein secondary structure on the ability to act as part of the molecular tape measure, an insertion of an artificial 164 amino acid sequence, Top7, was inserted between resides 140 & 141 of FliK and at the very C-terminus of FliK. The Top7 sequence folds into a stable globular structure that is too wide to fit through the central channel of the flagellum (Kuhlman et al., 2003). Thus, secretion of a FliK-Top7 chimera should not occur if Top7 folds prior to secretion and cannot be unfolded by the flagellar secretion apparatus. Cells expressing the FliK14O-Top7 chimera were phenotypically identical to a FliK null allele and produced polyhook structures. This suggests that Top7 folds to block secretion and prevent FliK from acting as a molecular tape measure. This result also suggests that flagellar-dependent type III secretion is not co-translational. Remarkably, cells producing Top7 fused to the C-terminal residue 405 of FliK did not produce any detectable flagellar structures suggesting that FliK secretion occurs during rod formation, but the C-terminal FliK-Top7 chimera irreversibly blocks the secretion channel preventing any significant structures from forming.

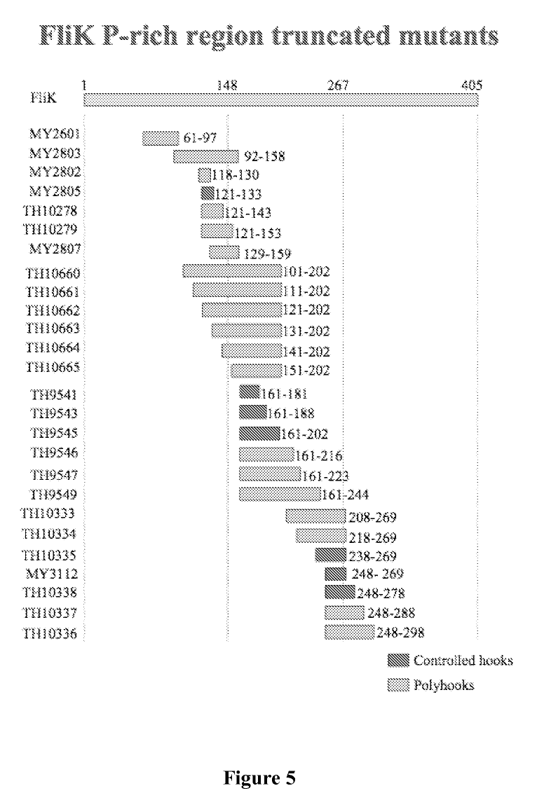

The proline-rich region of FliK was also examined for regions that could be deleted resulting in shorter hook structures. Twenty-six deletions of varying lengths were constructed (FIG. 5). The majority of deletions in FliK (nineteen) resulted in a FliK-null phenotype and produced polyhook structures (FIG. 5). However, seven deletions, Δ121-133, Δ161-181, Δ161-188, Δ161-202, Δ238-269, Δ238-269, Δ248-269, and Δ248-278 produced hook-filament structures with shortened hooks (Table 3). These results suggests that the proline-rich region has a role in the ability of FliK to interact with FlhB and flip the secretion-specificity switch, perhaps through allosteric effects on the C-terminal domain of FliK.

f) FliK is a Molecular Tape Measure for Rod-Length Determination in Polyrod Mutants

The polyrod mutations in FlgG appeared to loose an intrinsic rod-length control mechanism. However, introduction of a fliK null allele into a polyrod mutant strain resulted in super-polyrods of up to the length of the bacterial cell (1 μm). This result showed that rod-length in the polyrod mutant strains was controlled by FliK. The FliK length variants, which affected hook-length, were introduced into a FlgG* (polyrod) mutant strain. As with hook, the length of the polyrod was determined to increase or decrease with a corresponding increase or decrease in the length of FliK (Table 4). However, the FliK insertion variants increased rod length an average of only 0.11 nm per residue as compared to an increase in hook-length of 0.17 nm per residue. Thus FliK is a molecular tape measure for any rod or rod-hook basal structure, although the length of rod measured by FliK is shorter than the length of hook measured by FliK. This is probably a result of differences in polymerization rates between rod and hook subunits.

g) Modeling the FlgG Structure Based on Homology to FlgE

It seemed remarkable that the polyrod structures could result from single amino acid substitutions in FlgG. This showed a simple mechanism leading to the cessation of FlgG-rod growth and provided an important clue into the design of the flagellar structure. The amino acid sequence of FlgG was examined, and it was found that a high degree of amino acid identity with the flagellar hook protein (FlgE) (FIG. 6A) existed, which is assembled after FlgG-rod completion. There are two significant differences between the FlgG-rod and FlgE-hook sequences. First, the FlgG-rod has an insertion of 18 amino acids (residues 46 through 65 of FlgG) not present in FlgE where the majority of the polyrod mutations occurred (amino acids 52 through 66) (FIG. 6B). Second, FlgE-hook has a stretch of 146 amino acids in the middle of the protein that is not present in FlgG (FIG. 6A). The structure of FlgE has been determined (Samatey et al., 2004). The FlgE-hook protein contains two domains and the 146 amino acid region not present in FlgG-rod comprises a domain separated from the core domain by a hinge region The FlgG-rod sequence was modeled onto the FlgE-hook structure (FIG. 6C). Unfortunately, the first 70 amino acids of FlgE-hook were not structured, which corresponds to the first 90 amino acids in FlgG, and was where the majority of polyrod mutations were located (amino acids 52-68 of FlgG). However, two polyrod mutant sites that include the G183R/G183W and S197L mutations, reside close to each other at the very bottom of the predicted FlgG structural model, and two other polyrod mutant sites that include the D117Y and R132G mutations, are located close to each other in the middle of the structure (FIG. 6C). This allowed for the discovery of the mechanisms for FlgG stop-polymerization. That is, the 52-68 amino acid region and the amino acids at the bottom of the FlgG structure G183 and S197 interact to stop FlgG polymerization. This stacking of FlgG subunits likely results in a conformational change mediated through the region containing the D117 and R132 residues that prevents further FlgG polymerization.

h) Rod Length Control is Required for Normal P- and L-Ring Formation

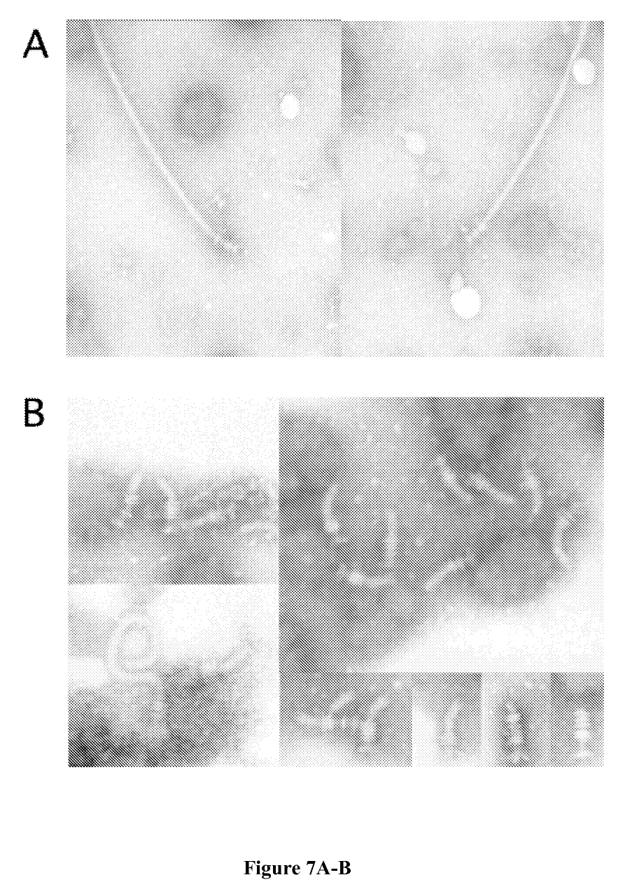

The polyrod mutants were originally isolated in a strain deleted for the P- and L-ring structural genes (ΔflgHI). Strains were constructed that only carried the FlgG* mutant alleles to see what effect the single FlgG* alleles might have on flagellar structure and assembly. Strains with only flgG* alleles were nonmotile, which was expected, but if subjected to flagella purification, intact flagellar structures were obtained with polyrods fused to filaments (FIG. 7A). Some FlgG* alleles, such as flgG*5664 (G53C), resulted in polyrods without any apparent hook structures (FIG. 7A). Other FlgG* alleles, such as flgG*5671 (P52L), resulted in polyrods with short hook structures attached. FIG. 7B shows isolated basal structures obtained after de-polymerization of the associated filaments. The rings associated with the basal structures varied from two to six per cell and appeared to depend on the length of the individual polyrods (FIG. 7B).

To determine the composition of the polyring structures a flgG* allele was introduced into separate flgI(P-ring) and flgH(L-ring) mutant strains. The flgG*flgH double mutant still produced polyrod-filament structures with polyrings, while the flgG*flhI double mutant produced only polyrods without rings or attached filaments (data not shown). Thus, the polyring structures can be multiple P-rings surrounding the polyrod structures.

i) Rod Length Control is Required for Outer Membrane Penetration of the Flagellum

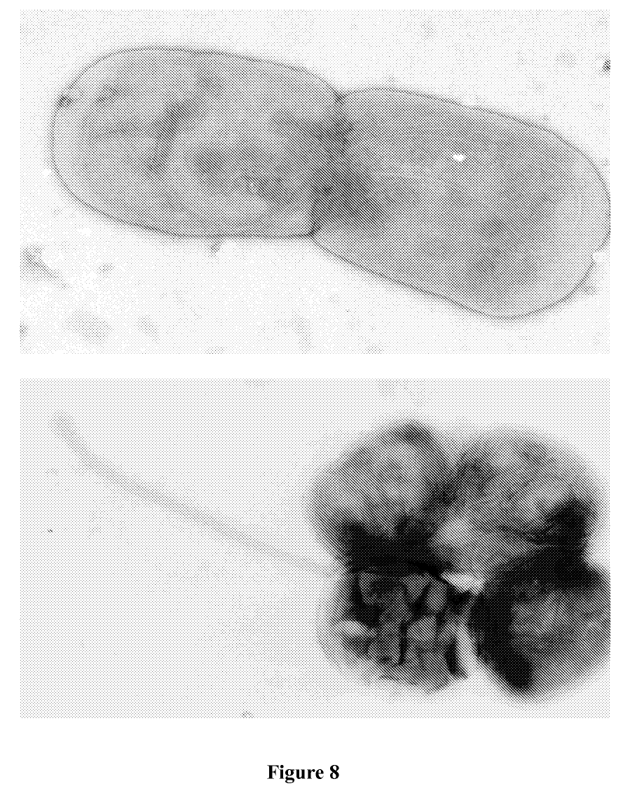

Although intact rod-filament structures could be obtained from strains with only the flgG* alleles, no visible flagellar filament structures were detected growing from the surfaces of these cells. However, upon closer examination after osmotic shock treatment, flagella could be visualized in the flgG* strains. Remarkably, filaments could be detected, but instead of growing outside the cell body, the flagella were growing between the inner and outer cell membranes (FIG. 8). Some isolated filaments were observed, but were always sheathed in membrane as seen in the bottom panel of FIG. 8. Curiously, similar structures have been reported to protrude from the spirochete Treponema phagedenis (Charon et al., 1992). Thus the polyrod mutant alleles produce flagella that were unable to penetrate the cell's outer membrane. These results show a simple mechanism for the evolution of spirochete flagella, which grow in the periplasmic space (Charon and Goldstein, 2002), by single amino acid changes in the FlgG rod subunits of gram-negative bacteria.

j) Discussion

An important regulatory checkpoint in flagellar assembly was demonstrated at the completion of the hook-basal body (HBB) structure. Upon HBB completion, there is a switch in the flagellar type III secretion substrate-specificity switch apparatus mediated by an interaction between the FliK ruler and an integral membrane component of the secretion system, FlhB. When the secretion at FlhB is flipped, the anti-σ²⁸ factor, FlgM is secreted and late flagellar gene transcription ensues. In this way FliC and FljB filaments are not produced until a HBB is assembled that they can polymerize onto. This study demonstrates another checkpoint in flagellar assembly prior to HBB completion. There is a checkpoint where 4 components of the flagellar assembly pathway intersect: (1) rod growth termination, (2) P- and L-ring formation, (3) hook polymerization and (4) outer membrane penetration. This checkpoint is initiated at the point of completion of the flagellar drive shaft mediated by a stop polymerization mechanism in the distal rod subunit FlgG. Upon rod completion, the transition to hook polymerization occurs and this is coupled to the formation of the P- and L-rings in the peptidoglycan and lipopolysaccharide layers, respectively and penetration of the outer membrane by the growing flagellum.

k) The Rod Stop-Polymerization Mechanism is Intrinsic to FlgG

The FlgG protein is the final component of the flagellar rod structure. Through a genetic selection that demanded for reduced FlgM protein in a flagellar mutant background lacking the P- and L-ring structures (ΔflgHI), mutants in the flgG locus were obtained. This same genetic selection resulted in loss-of-function mutations for the Fluke gene locus (flk). The Fluke protein was shown to prevent the premature secretion of FlgM in the P- and L-ring (ΔflgHI) mutant background. The goal of the genetic selection/screen devised in this study was to isolate Fluke-bypass mutations as a means to understand how Fluke prevented FlgM secretion through the uncompleted basal flagellar structures. The Fluke-bypass mutants in FlgG, flgG* alleles, were phenotypically identical to loss-of-function mutations in Fluke; they allowed FlgM secretion into the periplasm in the P- and L-ring (ΔflgHI) mutant background where it was degraded. However, they did this by losing an intrinsic mechanism that allowed the FlgG component of the rod to continue to polymerize beyond its normal length of 22 nm (Kubori et al., 1992) to longer structures that averaged 60 nm in length (FIG. 2C). A 3-dimensional model of the FlgG protein, based on homology with FlgE, whose structure has been solved (Samatey et al., 2004), shows that flgG* alleles are located in 3 regions of the FlgG protein. The majority of flgG* alleles were located to a region from residues 52 through 66 of FlgM. Significantly, two amino acids residues that include three flgG* alleles, G183R, G183W and S197L, are in close proximity to each other at the very base of the FlgG structure. Another two amino acid residues that include the G132R and D117Y flgG* alleles, are also in close proximity to each other in the middle of the structure and this region probably interacts with the 52-66 region. It is easy to visualize that the stacking of one FlgG subunit on another would allow an interaction between the 183 and 197 positions at the bottom of FlgG and the 52-66 amino acid region that would disrupt further FlgG polymerization.

l) The flgG* Phenotype is Dependent of the FliK Ruler and Functional Hook and Hook-Capping Genes

The phenotype of a Fluke-bypass flgG* allele was a result of FlgM secretion into the periplasm in the P- and L-ring (ΔflgHI) mutant background. This phenotype was dependent on the FliK ruler, and the presence of functional hook and hook-capping genes flgE and flgD, respectively. FlgM is a late secretion substrate and FlgM secretion occurs only after the flagellar type III secretion apparatus switches specificity from rod-hook type substrates to the late secretion substrates. The secretion specificity switch is dependent on interaction of FliK with the FlhB component on the flagellar secretion apparatus (Minamino et al., 2004). FliK is a rod-hook type secretion substrate and is secreted through the basal structure prior to hook completion (Minamino et al., 1999b). It has recently been shown that FliK interacts with the hook and hook-capping proteins (FlgE and FlgD) and these interactions are presumably required for FliK-dependent secretion substrate specificity switch (Moriya et al., 2006). The fact that functional fliK, flgE and flgD genes are required for the flgG* phenotype to allow secretion of FlgM into the periplasm in the ΔflgHI mutant background would support a role for interaction of FliK with FlgE and FlgD in making the secretion substrate-specificity switch.

m) FliK is a Molecular Tape Measure for the Length of the Basal Structure

The possibility that FliK could act as a molecular tape measure based on the analogous needle-length control mechanism by YscP in the Yersinia enterocolitica virulence-associated type III secretion system was tested. Insertions into FliK of amino acid regions from proteins that are secreted by type III systems, YscP, FlgM and FliC, were constructed. These insertions resulted in increased hook-length in other wise wild-type strains or increased rod length in the polyrod (flgG*) mutant strains. Regions of FliK were also found within the proline rich region that could be deleted yielding shorter hook structures. These results demonstrated that FliK does act to measure rod-hook length for structures with normal-length rods and hooks flgG⁺ or for polyrod (flgG*) structures. Because the FliK requires a functional hook and hook-capping gene even in the polyrod mutant strains to function as a molecular tape measure, and FliK has been shown to interact with FlgD and FlgE, it is likely that the polyrod polymerizes beneath a layer of hook and a layer of hook-capping proteins. Indeed, polyrods from some of the flgG* alleles had visible hook structures attached (FIG. 7B).

n) FliK is not a Simple Molecular Ruler

The FliK insertion variants increased hook length on an average of 0.17 n per residue. This is the amino acid spacing distance expected for a peptide-based molecular ruler that is alpha-helical in structure. The distance between amino acid residues in an alpha helix is 0.15 nm per residue and 0.4 nm per residue for a fully extended peptide (Nelson and Cox, 2000). The FliK insertion variants increased polyrod length on an average of 0.11 nm per residue. There is no peptide structure that compacts residues to a distance of 0.11 nm per residue. This argues against a molecular ruler mechanism. Recently, mutants in the flagellar hook protein were isolated that polymerized hook at a rate slower than wild-type hook subunits (Moriya et al., 2006). The result of expressing FlgE alleles with reduced polymerization rates was shorter, final hook structures. However, hooks of wild-type lengths were obtained for the polymerization-defective FlgE alleles if these alleles were over-expressed, while the over-expression of wild-type hook subunits resulted in longer than wild-type hook structures. This led to the hypothesis that hook-length was controlled by both a FliK measuring device and a molecular clock mechanism. The results presented here suggest that the rate of rod polymerization is about 65% the rate of hook polymerization and would account for an increase of only 0.11 nm per inserted residue in FliK compared to 0.17 nm per residue for hook-length. It is also consistent with over-lapping mechanisms of a FliK measure and molecular clock for both rod and hook length control.

A third mechanism proposed for a hook length control was a measuring cup device. The flagellar switch complex proteins assemble at the cytoplasmic base of the flagellum forming a cup-like structure known as the C-ring (FIG. 1A). Mutants in the switch complex proteins were isolated that resulted in hook-lengths ½ and ¾ the length of wild-type hook structures (Makishima et al., 2001). This led to the model that the C-ring, composed of about 30 subunits of FliG, 30 subunits of FliM and 90 subunits of FliN could contain affinity sites for the 120 hook subunits that make up the final hook structure. It was proposed that the emptying of the C-ring cup would determine the final hook length and provide access to FlhB in the membrane by FliK to flip the switch to late secretion substrates (Makishima et al., 2001). The main argument against this model is that mutants, defective in the hook-cap protein, FlgD, continue to secrete hook subunits and fail to switch to the late secretion mode (Minamino and Pugsley, 2005). However, it is possible that both FlgD and FlgE are associated with the measuring cup prior to hook assembly. Given the recent finding that the N-terminus of FliK binds to both FlgD and FlgE (Moriya et al., 2006), it is possible that FliK must also associate with FlgD and FlgE present in the cup prior to hook polymerization so that it is in physical proximity to be secreted immediately after hook polymerization to measure the rod-hook length and flip the switch at the correct time in flagellar assembly. Without FlgD, FliK might not associate with FlhB at the right time, consistent with the molecular clock mechanism described above, to flip the switch. It has also been proposed that localized translation occurs at the base of the flagellum (Karlinsey et al., 2000a). This mechanism would allow for the construction of multiple flagella in an individual bacterium at different times in the cell cycle. Instead of binding FlgE and FlgD protein subunits, the C-ring cup may contain RNA binding sites for mRNAs encoding flagellar structural genes. Thus, the switch complex mutants resulting in shorter hook structures, may lose a set of flgE-containing mRNA binding sites. This would result in reduced localized translation of FlgE subunits and could account for the quantum reduction (by ¼ or ½) observed in these mutants in the final lengths of the hooks. Fewer hook mRNA binding sites would result in reduced levels of translated hook protein and shorter hooks. This model is consistent with the molecular clock and FliK molecular tape measure mechanism. Also, this can account for the observation that over-expression of flgE-containing mRNA from mutlicopy plasmid vectors results in longer hook structures.

o) Model for FlgG Stop-Polymerization

The number of FlgG subunits at the distal end of the rod has been estimated to be about 26 subunits (Jones et al., 1990). Two stacks of FlgG subunits in the 11-protofilament flagellar structure would add up to 22 subunits, which is in close agreement to this estimate. We envision at least two mechanisms that would allow FlgG polymerization to stop. Both mechanisms would include interactions between residues Gly-183/Ser-197 and the 52-68 amino acid region. One mechanism predicts that the stacking of one FlgG residue onto another results in a conformational change of the second FlgG protein that prevents further interactions with additional FlgG subunits. The second mechanism predicts a slight twist might occur when one FlgG residue stacks on the other, resulting from the interactions between residues Gly-183/Ser-197 and the 52-68 amino acid region. The twist can be tolerated in one stack, but not in further stacking. The stacking models are supported by the fact that flgG* mutations appear to reside at the, top, bottom of the FlgG structure modeled to the homologous FlgE structure and a hinge region in the middle of the proposed structure (FIG. 6C).

p) A Role for FlgG Stop-Polymerization in P- and L-ring Formation and Outer Membrane Penetration by the Growing Flagellum: Implications for the Evolution for Spirochete Flagella

When the flgG* alleles were placed in strains competent for ring formation (flgH⁺ flgI⁺), multiple ring structures were observed surrounding the polyrods. In some cases, the flagella isolated from the flgG*flgH⁺ flgI⁺ strains had visible hook structures, only rod-filament structures. The polyrings remained in strains missing only the L-ring (flgG*flgI⁻ figI⁺), but all rings were absent in strains missing only the P-ring ring (flgG* flgH⁺ flgI⁻). This result was consistent with the formation of multiple P-rings on the polyrod structures and a lack of L-ring formation. These results suggest that the P-ring forms around FlgG two-layer stacks and the additional layers of FlgG allows for multiple layers of P-rings to form.

Another striking feature of the flgG* alleles in strains competent for ring formation (flgI⁺ flgI⁺), was the ability to form filaments on the polyrods and that the filaments failed to penetrate the outer membrane. Since the same phenotype was observed in a flgG* strain missing the L-ring, filament growth on the polyrod requires that only the P-ring is assembled. In wild-type HBB⁺ structures the P-rings are imbedded in the peptidoglycan layer at least 55 nm away from where filament polymerization begins.

The flagella of Salmonella can grow between the inner and outer membranes similar to what is seen in spirochete flagella. Many spirochete flagella lack P- and L-rings altogether (Charon and Goldstein, 2002). A BLAST search revealed that at least three species of spirochetes, Borrelia burgdorferi, Borrelia garnii and Leptospira interrogans possess P-rings, but none was found with a fliH gene to encode an L-ring. One remarkable result obtained in this study was the demonstration that evolution of a flagella form that protrudes from the cell surface as in Salmonella enterica to a flagella form that resides in the periplasm could occur by a single base change. Thus strong evidence was presented for a role in rod-length control and L-ring formation in S. enterica to allow the flagellum to penetrate the outer membrane and polymerize outside the cell.

q) Protein Nanotubes

Polyrod and super-polyrod structures can be utilized as protein nano-tubing, as the structures can be separated according to size. Unlike the hook, which is designed to be flexible, the rod is the rigid drive shaft of the flagellar motor. Alternatively, mutants that control cell body size can be employed to control the final length of the super-polyrod structures.

r) Experimental Procedures

Bacterial strains and plasmids. Bacterial strains used in this study originated from Salmonella enterica serovar Typhimurium strain LT2. FlgG is represented by SEQ ID NO: 1 (misslwiakt gldaqqtnmd viannlanvs tngfkrqrav fedllyqtir qpgaqsseqt tlpsglqigt gvrpvaterl hsqgnlsqtn nskdvaikgq gffqvmlpdg tsaytrdgsf qvdqngqlvt aggfqvqpai tipanalsit igrdgvvsvt qqgqaapvqv gqlnlftfinn dtglesigen lyietqssga pnestpglng agllyqgyve tsnvnvaeel vnmiqvqray einskavstt dqmlqkltql).

Media and standard genetic manipulations. Media, growth conditions, transductional methods, and motility assays were described (Aldridge et al., 2006). The generalized transducing phage of S. typhimurium P22 HT105/1 int-201 was used in all transductional crosses (Davis et al., 1980).

Genetic selection for polyrod mutants. Strain TH4987 (ΔflgHI958 motA5461::MudJ flhA^{ts}) carries a transcriptional fusion of the lac operon (MudJ) under expression of the σ²⁸-dependent motA promoter. It also carries an in-frame deletion of the P- and L-ring structural genes (ΔflgHI) and a temperature-sensitive mutation in an integral membrane component of the flagellar type III secretion apparatus (flhA^{ts}). This strain is Lac⁻ because in the absence of the P- and L-rings (ΔflgHI) the anti-σ²⁸ factor FlgM is not secreted from the cell, and σ²⁸-dependent transcription of the motA promoter (and thus the motA-lac operon reporter construct motA::MudJ) does not occur. At 30° C. the strain is FlhA⁺ resulting in a functional flagellar secretion apparatus, the flagellar rod structure forms and hook elongation initiates, but the structure fails to extend beyond the outer membrane due to the missing P- and L-ring (ΔflgHI) components. In strains defective only in P- and L-ring formation, loss of either FlgM or Fluke would result in σ²⁸-dependent transcription from the motA promoter (Lac⁺) (Karlinsey et al., 1997). At 42° C. the strain is FlhA⁻. The flagellar secretion apparatus is not functional without FlhA. Only loss of FlgM allows σ²⁸-dependent transcription in strains defective in FlhA (Karlinsey et al., 1997). To isolate Fluke-bypass mutants strain TH4987 was plated on minimal lactose (E-Lac) medium and incubated at 30° C. for 48 hr. The 30° C. Lac⁺ colonies were screened for those that were Lac⁻ at 42° C. by replica printing. The Lac^{ts} alleles allow σ²⁸-dependent motA transcription at 30° C. (FlgHI-negative FlhA⁺), but not at 42° C. (FlgHI-negative FlhA-negative). The Lac^{ts} mutants were screened for linkage to the flk locus by bacteriophage P22-mediated transduction to the flk region of the chromosome as described (Karlinsey et al., 1997). Those not linked to the flk region were kept as Fluke-bypass mutants (flk^{BP}). Insertions of transposon Tn10dTc linked to the flk^{BP} alleles followed by DNA sequence analysis was performed as described for the isolation of Tn10dTc linked to the serT locus (Chevance et al., 2005).

Electron microscopy. For visualization by electron microscopy, cells and isolated flagellar structures were stained with 1% phosphotungstic acid (PTA) (pH 7 or pH 5) and observed with a JEOL 1200Ex electron microscope at 80 kV.

FlgG structure modeling. A three-dimensional model of the flgG was constructed using the comparative modeling module of the PROTINFO server (http://www.protinfo.compbio.washington.edu), which has been shown to work well in the CASP protein structure prediction experiments (Hung et al., 2005; Hung and Samudrala, 2003). The structure of FlgE was used as a template for the modeling the flgG sequence excluding the N- and C-terminal insertions (Samatey et al., 2004). Images were produced using the Molscript (Kraulis, 1991) and Raster3D software (Merritt and Murphy, 1994).

### F. REFERENCES

Aizawa, S.-I. (1996). Flagellar assembly in Salmonella typhimurium. Mol Microbiol 20, 1-4.
Aldridge, P., and Hughes, K. T. (2002). Regulation of flagellar assembly. Curr Opin Microbiol 5, 160-165.
Aldridge, P., Karlinsey, J. E., Becker, E., Chevance, F. F. V., and Hughes, K. T. (2006). Flk prevents premature secretion of the anti-σ factor FlgM into the periplasm. Molec Microbiol 60, 630-642.
Armitage, J. P., Holland, I. B., Jenal, U., and Kenny, B. (2005). “Neural networks” in bacteria: making connections. J Bacteriol 187, 26-36.
Berg, H. C., and Anderson, R. A. (1973). Bacteria swim by rotating their flagellar filaments. Nature 245, 380-382.
Charon, N. W., and Goldstein, S. F. (2002). Genetics of motility and chemotaxis of a fascinating group of bacteria: the spirochetes. Annu Rev Genet. 36, 47-73.
Charon, N. W., Goldstein, S. F., Block, S. M., Curci, K., Ruby, J. D., Kreiling, J. A., and Limberger, R. J. (1992). Morphology and dynamics of protruding spirochete periplasmic flagella. J Bacteriol 174, 832-840.
Chevance, F. F. V., Karlinsey, J. E., Wozniak, C. E., and Hughes, K. T. (2005). A little gene with big effects: a serT mutant is defective in flgM gene translation. J Bacteriol 188, 297-304.
Chilcott, G. S., and Hughes, K. T. (2000). The coupling of flagellar gene expression to flagellar assembly in Salmonella typhimurium and Escherichia coli. Microbiol & Molec Biol Rev 64, 694-708.
Davis, R. W., Botstein, D., and Roth, J. R. (1980). Advanced Bacterial Genetics (Cold Spring Harbor, N.Y.: Cold Spring Harbor Laboratory).
Emerson, S. U., Tokuyasu, K., and Simon, M. I. (1970). Bacterial flagella: polarity of elongation. Science 169, 190-192.
Gillen, K. L., and Hughes, K. T. (1991a). Molecular characterization of flgM, a gene encoding a negative regulator of flagellin synthesis in Salmonella typhimurium. J Bacteriol 173, 6453-6459.
Gillen, K. L., and Hughes, K. T. (1991b). Negative regulatory loci coupling flagellin synthesis to flagellar assembly in Salmonella typhimurium. J Bacteriol 173, 2301-2310.
Hirano, T., Minamino, T., and Macnab, R. M. (2001). The role in flagellar rod assembly of the N-terminal domain of Salmonella FlgJ, a flagellum-specific muramidase. J Mol Biol 312, 359-369.
Hirano, T., Minamino, T., Namba, K., and Macnab, R. M. (2003). Substrate specificity classes and the recognition signal for Salmonella type III flagellar export. J Bacteriol 185, 2485-2492.
Hirano, T., Yamaguchi, S., Oosawa, K., and Aizawa, S.-I. (1994). Roles of FliK and FlhB in determination of flagellar hook length in Salmonella typhimurium. J Bacteriol 176, 5439-5449.
Hughes, K. T., Gillen, K. L., Semon, M. J., and Karlinsey, J. E. (1993). Sensing structural intermediates in bacterial flagellar assembly by export of a negative regulator. Science 262, 1277-1280.
Hung, L. H., Ngan, S. C., Liu, T., and Samudrala, R. (2005). PROTINFO: new algorithms for enhanced protein structure predictions. Nucleic Acids Res 33, W77-80.
Hung, L. H., and Samudrala, R. (2003). PROTINFO: Secondary and tertiary protein structure prediction. Nucleic Acids Res 31, 3296-3299.
Iino, T. (1969). Genetics and chemistry of bacterial flagella. Bacteriol Rev 33, 454-475.
Jones, C. J., Macnab, R. M., Okino, H., and Aizawa, S.-I (1990). Stoichiometric analysis of the flagellar hook-(basal-body) complex of Salmonella typhimurium. J Mol Biol 212, 377-387.
Journet, L., Agrain, C., Broz, P., and Cornelis, G. R. (2003). The needle length of bacterial injectisomes is determined by a molecular ruler. Science 302, 1757-1760.
Karlinsey, J. E., Lonner, J., Brown, K. L., and Hughes, K. T. (2000a). Translation/Secretion Coupling by Type III Secretion Systems. Cell 102, 487-497.
Karlinsey, J. E., Pease, A. J., Winkler, M. E., Bailey, J. L., and Hughes, K. T. (1997). The flk gene of Salmonella typhimurium couples flagellar P- and L-ring assembly to flagellar morphogenesis. J Bacteriol 179, 2389-2400.
Karlinsey, J. E., Shugo Tanaka, S., Bettenworth, V., Yamaguchi, S., Boos, W., Aizawa, S. I., and Hughes, K. T. (2000b). Completion of the hook-basal body of the Salmonella typhimurium flagellum is coupled to FlgM secretion and fliC transcription. Mol Microbiol 37, 1220-1231.
Kojima, S., and Blair, D. (2004). The bacterial flagellar motor: structure and function of a complex molecular machine. Int Rev Cytol 233, 93-134.
Kraulis, P. J. (1991). MOLSCRIPT: A program to produce both detailed and schematic plots of protein structures. J Appl Crystal 24, 946-950.
Kubori, T., Shimamoto, N., Yamaguchi, S., Namba, K., and Aizawa, S. (1992). Morphological pathway of flagellar assembly in Salmonella typhimurium. J Mol Biol 226, 433-446.
Kuhlman, B., Dantas, G., Ireton, G. C., Varani, G., Stoddard, B. L., and Baker, D. (2003). Design of a novel globular protein fold with atomic-level accuracy. Science 302, 1364-1368.
Kutsukake, K. (1994). Excretion of the anti-sigma factor through a flagellar substructure couples flagellar gene expression with flagellar assembly in Salmonella typhimurium. Mol Gen Genet. 243, 605-612.
Kutsukake, K., Minamino, T., and Yokoseki, T. (1994). Isolation and characterization of FliK-independent flagellation mutants from Salmonella typhimurium. J Bacteriol 176, 7625-7629.
Macnab, R. M. (1996). Flagella and Motility, In Escherichia coli and Salmonella typhimurium:Cellular and Molecular Biology, F. C. Neidhart, et al., ed. (Washington, D.C.: American Society for Microbiology), pp. 123-145.
Macnab, R. M. (1999). The bacterial flagellum: reversible rotary propellor and type III export apparatus. J Bacteriol 181, 149-153.
Macnab, R. M. (2004). Type III flagellar protein export and flagellar assembly. Biochim Biophys Acta 1694, 207-217.
Makishima, S., Komoriya, K., Yamaguchi, S., and Aizawa, S. I. (2001). Length of the flagellar hook and the capacity of the type III export apparatus. Science 291, 2411-2413.
Merritt, E. A., and Murphy, M. E. (1994). Raster3D Version 2.0. A program for photorealistic molecular graphics. Acta Crystallogr D Biol Crystallogr 50, 869-873.
Minamino, T., Doi, H., and Kutsukake, K. (1999a). Substrate specificity switching of the flagellum-specific export apparatus during flagellar morphogenesis in Salmonella typhimurium. Biosci Biotechnol Biochem 63, 1301-1303.
Minamino, T., Gonzalez-Pedrajo, B., Yamaguchi, K., Aizawa, S.-I., and Macnab, R. M. (1999b). FliK, the protein responsible for flagellar hook length control in Salmonella, is exported during hook assembly. Mol Microbiol 34, 295-304.
Minamino, T., and Macnab, R. M. (1999). Components of the Salmonella flagellar export apparatus and classification of export substrates. J Bacteriol 181, 1388-1394.
Minamino, T., and Namba, K. (2004). Self-assembly and type III protein export of the bacterial flagellum. J Mol Microbiol Biotechnol 7, 5-17.
Minamino, T., and Pugsley, A. P. (2005). Measure for measure in the control of type III secretion hook and needle length. 56, 303-308.
Minamino, T., Saijo-Hamano, Y., Furukawa, Y., Gonzalez-Pedrajo, B., Macnab, R. M., and Namba, K. (2004). Domain organization and function of Salmonella FliK, a flagellar hook-length control protein. J Mol Biol 341, 491-502.
Minamino, T., Yamaguchi, S., and Macnab, R. M. (2000). Interaction between FliE and FlgB, a proximal rod component of the flagellar basal body of Salmonella. J Bacteriol 182, 3029-3036.
Moriya, N., Minamino, T., Hughes, K. T., Macnab, R. M., and Namba, K. (2006). The type III flagellar export specificity switch is dependent on FliK ruler and a molecular clock. J Mol Biol 359, 466-477.
Nelson, D. L., and Cox, M. M. (2000). Lehninger Principles of Biochemistry, 3rd edn (New York: Worth Publishers).
Ohnishi, K., Kutsukake, K., Suzuki, H., and Iino, T. (1992). A novel transcriptional regulatory mechanism in the flagellar regulon of Salmonella typhimurium: an anti sigma factor inhibits the activity of the flagellum-specific sigma factor, sF. Mol Microbiol 6, 3149-3157.
Ohnishi, K., Ohto, Y., Aizawa, S.-I., Macnab, R. M., and Iino, T. (1994). FlgD is a scaffolding protein needed for flagellar hook assembly in Salmonella typhimurium. J Bacteriol 176, 2272-2281.
Okino, H., Isomura, M., Yamaguchi, S., Magariyama, Y., Kudo, S., and Aizawa, S. I. (1989). Release of flagellar filament-hook-rod complex by a Salmonella typhimurium mutant defective in the M ring of the basal body. J Bacteriol 171, 2075-2082.
Patterson-Delafield, J., Martinez, R. J., Stocker, B. A., and Yamaguchi., S. (1973). A new fla gene in Salmonella typhimurium—flaR—and its mutant phenotype—superhooks. Arch Mikrobiol 90, 107-120.
Samatey, F. A., Matsunami, H., Imada, K., Nagashima, S., Shaikh, T. R., Thomas, D. R., Chen, J. Z., Derosier, D. J., Kitao, A., and Namba, K. (2004). Structure of the bacterial flagellar hook and implication for the molecular universal joint mechanism. Nature 431, 1062-1068.
Yonekura, K., Maki, S., Morgan, D. G., DeRosier, D. J., Vonderviszt, F., Imada, K., and Namba, K. (2000). The bacterial flagellar cap as the rotary promoter of flagellin self-assembly. Science 290, 2148-2152.

What is claimed is:

1. A composition comprising an engineered bacterial flagella that is greater in length than a flagella from a naturally occurring organism.

2. The composition of claim 1, wherein the engineered flagella is about 3 times greater in length.

3. The composition of claim 1, wherein the engineered flagella is greater than about 0.5 μm.

4. The composition of claim 1, wherein the engineered flagella is greater than about 1.0 μm.

5. The composition of claim 1, wherein the engineered flagella forms a helix.

6. The composition of claim 1, wherein the engineered bacterial flagella comprises a mutation in FlgG protein.

7. The composition of claim 6, wherein the mutation in the FlgG protein is found in amino acid residues 52-68 of SEQ ID NO: 1.

8. The composition of claim 7, wherein the mutation is a single amino acid residue.

9. The composition of claim 7, wherein the mutation comprises more than one amino acid residue.

10. The composition of claim 1, wherein the engineered bacterial flagella comprises a nanotube.

11. The composition of claim 10, wherein the nanotube also comprises a binding agent having an affinity for a biomolecule of interest coupled to the surface of the nanotube.

12. The composition of claim 11, wherein the nanotube further comprises a reporter molecule coupled to the surface of the nanotube, wherein the reporter molecule is capable of providing a signal.

13. The composition of claim 11, wherein the biomolecule of interest is selected from at least one of the following: a polypeptide, a protein, a glycoprotein, a nucleic acid, a carbohydrate, a lipid, a vitamin, a virus, a virus infected cell, and combinations thereof.

14. The composition of claim 11, wherein the biomolecule comprises an RNA or DNA virus.

15. The composition of claim 11, wherein the binding agent is selected from one of the following: a metal, a plastic, a polymer, a polynucleotide, a polypeptide, a protein, an amino acid, a glycoprotein, a lipid, a carbohydrate, a fatty acid, a fatty ester, a macromolecular polypeptide complex, and a combination thereof.

16. The composition of claim 15, wherein the amino acid comprises histidine.

17. A vector comprising flgG, wherein flgG comprises a mutation that allows for the formation of a polyrod.

18. The vector of claim 1, wherein the mutation comprises a mutation in one or more amino acid residues 52-68 of SEQ ID NO: 1.

19. The vector of claim 18, wherein the vector further comprises a mutation in fliK.

20. A cell expressing the vector of claim 19.

21. A system comprising the cell of claim 20.

22. An organism comprising the cell of claim 20.

23. A method of producing a polyrod, comprising:
a) introducing into a cell the vector of claim 17;
b) expressing the polypeptide encoded by the vector of step a); thereby producing a polyrod.

24. The method of claim 23, wherein the cell further expresses proteins needed for producing flagella.

25. The method of claim 23, wherein the polyrod can be used as a nanotube.

26. The method of claim 25, wherein the nanotube also comprises a binding agent having an affinity for a biomolecule of interest coupled to the surface of the nanotube.

27. The method of claim 26, wherein the nanotube further comprises a reporter molecule coupled to the surface of the nanotube, wherein the reporter molecule is capable of providing a signal.

28. The method of claim 25, wherein the biomolecule of interest is selected from at least one of the following: a polypeptide, a protein, a glycoprotein, a nucleic acid, a carbohydrate, a lipid, a vitamin, a virus, a virus infected cell, and combinations thereof.

29. The method of claim 25, wherein the biomolecule comprises an RNA or DNA virus.

30. The method of claim 25, wherein the binding agent is selected from one of the following: a metal, a plastic, a polymer, a polynucleotide, a polypeptide, a protein, an amino acid, a glycoprotein, a lipid, a carbohydrate, a fatty acid, a fatty ester, a macromolecular polypeptide complex, and a combination thereof.

31. The method of claim 30, wherein the amino acid comprises histidine.

32. The method of claim 31, wherein histidine residues are produced by inserting his codons into the vector.

33. A transgenic organism that produces a bacterial flagella greater in length than a naturally occurring flagella.

34. The transgenic organism of claim 33, wherein the organism is a bacterium.

35. The transgenic organism of claim 33, wherein the flagella is about 3 times greater in length.

36. The transgenic organism of claim 33, wherein the flagella is greater than about 0.5 μm.

37. The transgenic organism of claim 33, wherein the flagella is greater than about 1.0 μm.

38. The transgenic organism of claim 33, wherein the organism comprises a mutation in FlgG protein.

39. The transgenic organism of claim 33, wherein the mutation in the FlgG protein is found in amino acid residues 52-68 of SEQ ID NO: 1.

40. The transgenic organism of claim 39, wherein the mutation is a single amino acid residue.

41. The transgenic organism of claim 39, wherein the mutation comprises more than one amino acid residue.

42. The transgenic organism of claim 33, wherein the engineered bacterial flagella comprises a nanotube.

43. The transgenic organism of claim 42, wherein the nanotube further comprises a binding agent having an affinity for a biomolecule of interest coupled to the surface of the nanotube.

44. The transgenic organism of claim 42, wherein the nanotube further comprises a reporter molecule coupled to the surface of the nanotube, wherein the reporter molecule is capable of providing a signal.

45. The transgenic organism of claim 42, wherein the biomolecule of interest is selected from at least one of the following: a polypeptide, a protein, a glycoprotein, a nucleic acid, a carbohydrate, a lipid, a vitamin, a virus, a virus infected cell, and combinations thereof.

46. The transgenic organism of claim 42, wherein the biomolecule comprises an RNA or DNA virus.

47. The transgenic organism of claim 42, wherein the binding agent is selected from one of the following: a metal, a plastic, a polymer, a polynucleotide, a polypeptide, a protein, an amino acid, a glycoprotein, a lipid, a carbohydrate, a fatty acid, a fatty ester, a macromolecular polypeptide complex, and a combination thereof.

48. The transgenic organism of claim 47, wherein the amino acid comprises histidine.

49. A cell comprising a mutation in flgG that allows the flagella to grow longer than a cell with wild type flgG.

50. The cell of claim 49, wherein the cell is a eukaryote.

51. The cell of claim 49, wherein the cell is a prokaryote.

52. The cell of claim 51, wherein the cell is a bacterium.

53. The cell of claim 52, wherein the bacterium is Salmonella.

54. The cell of claim 49, wherein the flagella is about 3 times greater in length.

55. The cell of claim 49, wherein the flagella is greater than about 0.5 μm.

56. The cell of claim 34, wherein the flagella is greater than about 1.0 μm.

57. The cell of claim 49, wherein the mutation in the nucleic acid corresponds to a mutation in SEQ ID NO: 1.

58. The cell of claim 49, wherein the mutation is in amino acid region 52-68 of SEQ ID NO: 1.

59. The cell of claim 58, wherein the mutation is a single amino acid residue.

60. The cell of claim 58, wherein the mutation comprises more than one amino acid residue.

61. The cell of claim 49, wherein the flagella comprises a polyrod.

62. The cell of claim 61, wherein the polyrod comprises a nanotube.

63. The cell of claim 62, wherein the nanotube further comprises a binding agent having an affinity for a biomolecule of interest coupled to the surface of the nanotube.

64. The cell of claim 62, wherein the nanotube further comprises a reporter molecule coupled to the surface of the nanotube, wherein the reporter molecule is capable of providing a signal.

65. The cell of claim 62, wherein the biomolecule of interest is selected from at least one of the following: a polypeptide, a protein, a glycoprotein, a nucleic acid, a carbohydrate, a lipid, a vitamin, a virus, a virus infected cell, and combinations thereof.

66. The cell of claim 62, wherein the biomolecule comprises an RNA or DNA virus.

67. The cell of claim 62, wherein the binding agent is selected from one of the following:
a metal, a polymer, a plastic, a polynucleotide, a polypeptide, a protein, an amino acid, a glycoprotein, a lipid, a carbohydrate, a fatty acid, a fatty ester, a macromolecular polypeptide complex, and a combination thereof.

68. The cell of claim 67, wherein the amino acid comprises histidine.

69. A method of sequencing comprising using the nanotube of claim 10.


SEQUENCE LISTING (1 sequence)

SEQ ID NO: 1 - protein, 260 aa, Artificial Sequence
  Description of Artificial Sequence; note = synthetic construct
MISSLWIAKTGLDAQQTNMDVIANNLANVSTNGFKRQRAVFEDLLYQTIRQPGAQSSEQTTLPSGLQIGTGVRPVATERLHSQGNLSQTNNSKDVAIKGQGFFQVMLPDGTSAYTRDGSFQVDQNGQLVTAGGFQVQPAITIPANALSITIGRDGVVSVTQQGQAAPVQVGQLNLTTFMNDTGLESIGENLYIETQSSGAPNESTPGLNGAGLLYQGYVETSNVNVAEELVNMIQVQRAYEINSKAVSTTDQMLQKLTQL